(19) United States
(12) Patent Application Publication
(10) Pub. No.: US 2006/0069038 A1
(43) Pub. Date: Mar. 30, 2006

(54) Irreversible caspase-3 inhibitors as active site probes

(76) Inventors: John Colucci, Montreal (CA); Andre Giroux, Quebec (CA); Yongxin Han, Quebec (CA); Nathalie Methot, Montreal (CA); Donald W Nicholson, Quebec (CA); Sophie Roy, Quebec (CA); John Paul Vaillancourt, Quebec (CA); Paul Tawa, Quebec (CA)

Correspondence Address: MERCK AND CO., INC, P O BOX 2000, RAHWAY, NJ, 07065-0907, US

(21) Appl. No.: 10/542,684

(22) PCT Filed: Feb. 5, 2004
(86) PCT No.: PCT/CA04/00152

§ 371 Date: Jul. 19, 2005

Related U.S. Application Data
(60) Provisional application No. 60/445,560, filed on Feb. 7, 2003.

(51) Int. Cl. A61K 38/04; A61K 31/4188; C07K 5/04

(52) U.S. Cl. 514/19; 514/383; 548/304.1; 562/450

(57) ABSTRACT

The present invention encompasses a compound of Formula (I) useful as caspase active site probes. These probes can be used to determine whether a caspase has been activated, in cells or in tissues of animal models of various pathologies. Furthermore, through competition based assays, these caspase active site probes can be used to calculate the percentage of occupancy of active caspases by other, unlabeled inhibitors.

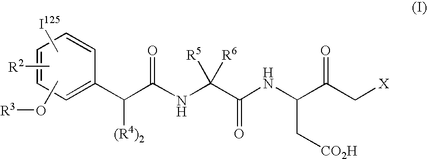

### BACKGROUND OF THE INVENTION

Apoptosis is a form of cellular death in which a cell is disassembled in an organized and orderly fashion. Apoptosis, in most instances is a normal process necessary for cellular homeostasis. A number of pathologies, however, exhibit abnormal cell death, with either too much or too little. It is therefore of high interest to modulate apoptosis with pharmacological agents and achieve improvement of patient health.

Many of the cellular morphological changes encountered during apoptosis are brought about by cysteine proteases termed caspases. These enzymes exist in all cell types in a dormant form and are activated through proteolytic processing following initiation of the apoptotic program. Active caspases consist of heterotetramers of two 20 kDa and two 10 kDa subunits with the active site cysteines located on the 20 kDa subunits. Caspases invariably cleave their protein substrates after an aspartic acid residue preceded by a short and relatively conserved consensus sequence. Most pharmacological inhibitors of caspases are short peptidomimetic molecules containing aspartic acid, and have been shown to inhibit apoptosis in cells. An important consideration for therapies relying on caspase inhibition the percentage of caspase active site that needs to be occupied by an inhibitor in order to achieve therapeutic benefit. The present invention describes labeled caspase probes that bind irreversibly to the active site cysteine. These probes enable us to determine whether a caspase has been activated in cells or in tissues of animal models of various pathologies. Furthermore, through competition-based assays, these caspase active site probes allow us to calculate the percentage of occupancy of active caspases by other, unlabeled inhibitors.

### SUMMARY OF THE INVENTION

The present invention encompasses a compound of Formula I 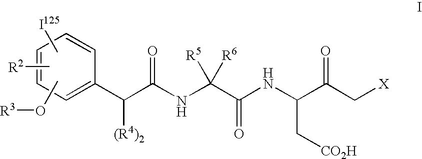
useful as caspase active site probes. These probes can be used to determine whether a caspase has been activated, in cells or in tissues of animal models of various pathologies. Furthermore, through competition based assays, these caspase active site probes can be used to calculate the percentage of occupancy of active caspases by other, unlabeled inhibitors.

### BRIEF DESCRIPTION OF THE DRAWINGS

FIG. 1 Structure of [¹²⁵I]-M808 and inhibitory constants (Kᵢ) against a subset of caspases.

FIG. 2. [¹²⁵I]-M808 detects active caspases in protein extracts. The large subunit of fully processed caspase-3 migrates at 17 kDa (p17). Partially processed caspase-3 retains part (p19) or all (p20) of the pro-domain, but is as active as the p17 form. Removal of the prodomain is self-catalyzed. A-As expected, the p17 subunit of purified recombinant human caspase-3 covalently bound [¹²⁵]-M808 (lane 1). All granzyme-B treated rat tissues contained a [125I]-M808-labeled 19 kDa protein, while liver and thymus showed an additional polypeptide migrating at 20 kDa (lanes 2-5). Inclusion of M791 during labeling reaction eliminates or strongly reduces the p17 and p19-labeled bands (lanes 6-9). B-Intensity of p17 and p19 [¹²⁵I] M808-labeled proteins correlates with total amount of caspase-3 like activity present in granzyme-B treated tissue extracts. C-[¹²⁵I]-M808 labels p17, p19 and p20 caspase subunits in apoptotic NT2 and Jurkat cells, but not healthy cells. D-[¹²⁵I]-M808 labels p17 caspase subunit in 3 different models of cellular injury (HI; Hypoxia-Ischemia, MI; Myocardial infarct; CLP; Cecal Ligation and puncture for sepsis. Ipsilateral, VLV (ventral left ventricle) and sham are uninjured, control tissues).

FIG. 3. Potency assessment caspase inhibitors by [¹²⁵]-M808 labeling and by DEVD-AMC cleavage activity. A, B-Caspase inhibitor titration and [¹²⁵]-M808 labeling with purified recombinant human caspase-3. A-M791 with 5 minute labeling reaction and M791 with 60 minute labeling reaction. In A, the indicated IC₅₀ was defined as the concentration of M791 that reduced the intensity of [¹²⁵I]-M808-labeled p17 subunit of caspase-3 by 50%. B-Caspase inhibitor titration and DEVD-AMC cleavage activity with recombinant human caspase-3 that had been incubated with indicated concentration of M791. Under the fluorogenic assay conditions Kᵢ=IC₅₀/2.

FIG. 4. Detection of active caspases with [¹²⁵I]-M808 in living cells. A-Caspase-3 western blot on camptothecin-treated Jurkat cell extracts. B-[¹²⁵I]-M808-labeled p17, p19 and p20 caspase subunits in apoptotic Jurkat cells exposed to increasing amounts of M791′ C-DNA fragmentation activity (subdiploid cell population) in apoptotic Jurkat cells treated with increasing amounts of M791.

FIG. 5. Determination of caspase active site occupancy by M867 in cultured rat thymocytes. A-Theoretical calculations for the determination of the percentage of caspase-3 active sites occupied by M867 in whole cells. The total amount of caspase-3 p17 subunit is determined densitometrically by either Western blotting of saturation [¹²⁵I]-M808 labeling. Partial labeling of active caspases in whole cells is perforemd (see materials and methods). A graph of the p17 amount against partial [¹²⁵I]-M808 labeling signal in the absence of M867 (or other reversible caspase inhibitors) is plotted. The [¹²⁵I]-M808 signal expected from the measured p17 amount is estimated form this plot. The actual [¹²⁵I]-M808 signal (by partial labeling) in the presence of reversible inhibitor is measured. The ratio of actual [¹²⁵I]-M808 signal over expected [¹²⁵I]-M808 signal multiplied by 100=the percentage of caspase active sites unoccupied by an inhibitor. The percentage occupancy=100−% free active sites. B-C An actual determination of caspase active site occupancy by M867 in whole rat thymocytes. B-caspase-3 western blot. C-[¹²⁵I]-M808 signal from whole rat thymocytes exposed to increasing concentrations of M867. Comparison of M867 caspase inhibition potency (IC50) determined by DNA fragmentation inhibition and [¹²⁵I]-M808 labeling is also depicted.

FIG. 6. A-Schematic representation of in vivo labeling of active caspases with [¹²⁵I]-M808 in CLP-operated mice. Mice underwent either sham surgery (n=2) or CLP surgery (n=5). [¹²⁵I]-M808 was injected intravenously 23.15 h later, and animals were euthanized 24 h post-surgery. B-Caspase-3 western blot of mouse thymi extracts following CLP and administration of [¹²⁵I]-M808. Arrows indicate full length pro-caspase-3 (p32) and the processed caspase large subunit (p17). C— in-vivo [¹²⁵I]-M808-labeled protein in thymi extracts from septic mice. Arrow points to a radiolabeled caspase large subunit.

FIG. 7. Comparison of p17 caspase labeled with [¹²⁵I]-M808 for a short or a long incubation period. Septic rats were dosed intravenously with either vehicle or the indicated concentration of M867. Thymi were collected 24 hours post-CLP surgery. Western blot showing the appearance of cleaved p17 caspase-3. Partial caspase-3 labeling with high specific activity probe for 5 minutes. Saturated [¹²⁵I]-M808 labeling of active caspases in the same thymi extracts with low specific activity probe for 3 hours or 18 hours. During the long incubation period, M867 originally present on caspase-3 (seen from low or absent labeling) dissociates from the active site and is replaced by the irreversible active site probe [¹²⁵I]-M808. Displacement is essentially complete by 3 hours since no change in labeling intensity was observed between a 3 h and 18 h incubation period at 37° C.

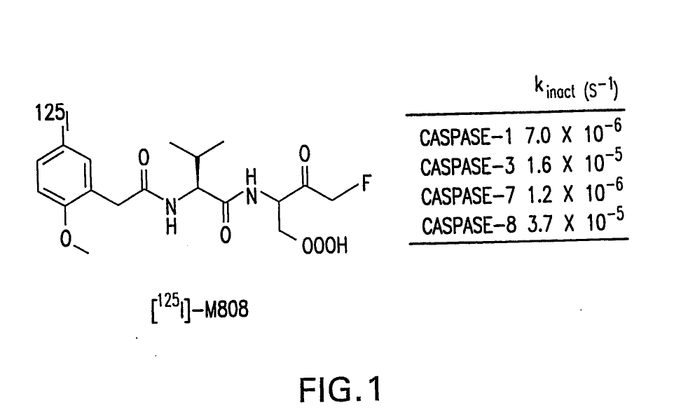

### DETAILED DESCRIPTION OF THE INVENTION

The invention encompasses a compound represented by Formula I: 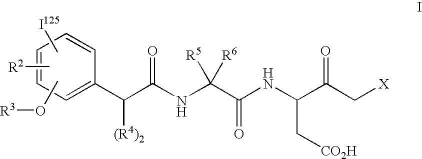
or a salt, ester or hydrate thereof, wherein:
X is halo, or
X is O—W-Z, wherein W is a bond, —CH₂—, (O)— or —C(O)CH₂—;
Z is selected from the group consisting of:
   (1) H,
   (2) C₁₋₁₁alkyl,
   (3) C₃₋₁₁cycloalkyl or a benzofused analog thereof,
   (4) phenyl or naphthyl, and
   (5) HET¹, wherein HET¹ represents a 5- to 10-membered mono- or bicyclic, aromatic or non-aromatic ring, or a benzofused analog thereof, containing 1-3 heteroatoms selected from O, S and N,
groups (2), (3) and (5) above are optionally substituted with 1-2 oxo groups, groups (2)-(5) above are further optionally substituted with 1-3 substituents independently selected from the group consisting of:
   (a) halo
   (b) nitro,
   (c) hydroxy,
   (d) C₁₋₄alkyl,
   (e) C₁₋₄alkoxy,
   (f) C₁₋₄alkylthio,
   (g) C₃₋₆cycloalkyl,
   (h) phenyl or naphthyl,
   (i) phenoxy,
   (j) benzyl,
   (k) benzyloxy, and
   (l) a 5 or 6-membered aromatic or non-aromatic ring containing from 1-3 heteroatoms selected from O, S and N,
groups (d)-(g) above are optionally substituted with oxo and 1-3 substituents independently selected from halo and C₁₋₄alkoxy,
groups (h)-(l) above are optionally substituted with 1-3 substituents independently selected from halo and C₁₋₄alkyl, and
group (4) is further optionally substituted up to its maximum with halo groups;
R² is selected from the group consisting of:
   (1) H,
   (2) halo,
   (3) hydroxy,
   (4) nitro,
   (5) cyano,
   (6) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo or carboxy groups and further optionally substituted with 1-3 substituents independently selected from the group consisting of:
   (a) halo,
   (b) hydroxy
   (c) cyano,
   (d) C₁₋₄alkoxy,
   (e) —NHR⁷, wherein R⁷ is independently H or C₁₋₅alkyl,
   (f) —S(O)₀₋₂C₁₋₄alkyl, and
   (g) HET², wherein HET² represents a 5- to 7-membered aromatic or non-aromatic ring containing 14 heteroatoms selected from O, S and NR⁸, wherein R⁸ is independently H or C₁₋₅alkyl, said HET² being optionally substituted with oxo and further optionally substituted with 1-2 substituents independently selected from halo and C₁₋₄alkyl, said C₁₋₄alkyl being optionally substituted with 1-3 halo groups,
   (7) phenoxy or —S(O)₀₋₂phenyl,
   (8) benzyloxy or —S(O)₀₋₂benzyl,
   (9) benzoyl,
   (10) phenyl or naphthyl,
   (11) —O-HET² or S-HET², said HET² being optionally substituted with oxo and further optionally substituted as defined below, and
   (12) HET³, wherein HET³ is a 5- or 6-membered aromatic or non-aromatic ring, or a benzofused analog thereof, containing from 1 to 4 heteroatoms selected from O, S and N, said HET³ being optionally substituted with oxo and further optionally substituted as defined below,
groups (7)-(12) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups;
R³ is phenyl or C₁₋₁₀alkyl, said C₁₋₁₀alkyl optionally substituted with 1-2 oxo or carboxy groups and further optionally substituted with 1-3 substituents independently selected from the group consisting of:
   (a) halo,
   (b) hydroxy
   (c) cyano,
   (d) C₁₋₄alkoxy,
   (e) —NHR⁷, wherein R⁷ is independently H or C₁₋₅alkyl,
   (f) —S(O)₀₋₂C₁₋₄alkyl, and
   (g) HET², wherein HET² represents a 5- to 7-membered aromatic or non-aromatic ring containing 14 heteroatoms selected from O, S and NR⁸, wherein R⁸ is independently H or C₁₋₅alkyl, said HET² being optionally substituted with oxo and further optionally substituted with 1-2 substituents independently selected from halo or C₁₋₄alkyl, said C₁₋₄alkyl being optionally substituted with 1-3 halo groups,
each R⁴ is independently selected from the group consisting of: H, halo, hydroxy, C₁₋₆alkyl and C₁₋₄alkoxy, said C₁₋₆alkyl and C₁₋₄alkoxy being optionally substituted with oxo and further optionally substituted with 1-3 halo groups; and
R⁵ is selected from the group consisting of: H, phenyl, naphthyl, C₁₋₆alkyl optionally substituted with OR¹² and 1-3 halo groups, and C₅-7 cycloalkyl optionally containing one heteroatom selected from O, S and NR¹³,
wherein R¹² is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy, and
R¹³ is H or C₁₋₄alkyl optionally substituted with 1-3 halo groups; and
R⁶ represents H;
or in the alternative, R⁵ and R⁶ are taken in combination and represent a ring of 47 members, said ring optionally containing one heteroatom selected from O, S and NR¹³.

An embodiment of the invention encompasses a compound of Formula I wherein X is halo.

Another embodiment of the invention encompasses a compound of Formula I wherein X is —O—W-Z. Within this embodiment Z is selected from the group consisting of:
(1) C₁₋₁₁alkyl,
(2) C³⁻¹¹cycloalkyl or a benzofused analog thereof, and
(3) phenyl or naphthyl,
   wherein groups (1)-(3) above are optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₁₄alkylthio,
(g) C₃₋₆cycloalkyl,
(h) phenyl or naphthyl,
(i) phenoxy,
(j) benzyl and
(k) benzyloxy.

Another embodiment of the invention encompasses a compound of Formula I wherein R³ is methyl.

Another embodiment of the invention encompasses a compound of Formula I wherein R² and each R⁴ are hydrogen.

Another embodiment of the invention encompasses a compound of Formula I wherein R⁵ is selected from the group consisting of: C₁₋₆alkyl, phenyl and naphthyl.

Another embodiment of the invention encompasses a compound of Formula I wherein:
X is halo or —W-Z;
W is a bond, —CH₂—, —C(O)— or —C(O)CH₂—;
Z is selected from the group consisting of:
   (1) C₁₋₆alkyl, optionally substituted with 1-3 halo groups,
   (2) C₃₋₁₁cycloalkyl or a benzofused analog thereof, and
   (3) phenyl or naphthyl, optionally substituted with 1-3 groups independently selected from halo or C₁₋₄alkyl,
R³ is methyl, ethyl or phenyl;
R² and each R⁴ are hydrogen;
R⁵ is selected from the group consisting of: C₁₋₆alkyl, C₅₋₇cycloialkyl, phenyl and naphthyl; and
R6 is hydrogen.

Another embodiment of the invention encompasses a compound of Formula II 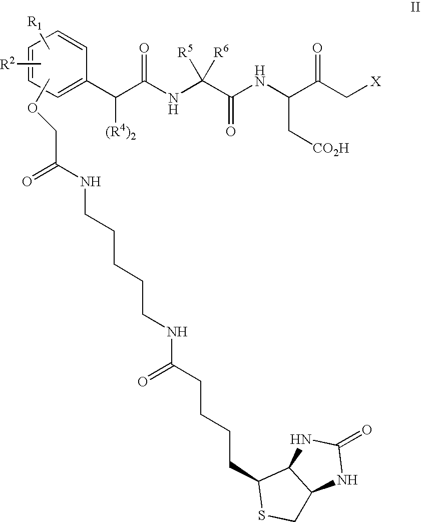
or a salt, ester or hydrate thereof, wherein:
X is halo;
R¹ and R² are each independently selected from the group consisting of:
   (1) H,
   (2) halo,
   (3) hydroxy, (4) nitro,
   (5) cyano,
   (6) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo or carboxy groups and further optionally substituted with 1-3 substituents independently selected from the group consisting of:
      (a) halo,
      (b) hydroxy
      (c) cyano,
      (d) C₁₋₄alkoxy,
      (e) —NHR⁷, wherein R⁷ is independently H or C₁₋₅alkyl,
      (f) —S(O)₀₋₂C₁₋₄alkyl, and
      (g) HET², wherein HET² represents a 5- to 7-membered aromatic or non-aromatic ring containing 14 heteroatoms selected from O, S and NR⁸, wherein R⁸ is independently H or C₁₋₅alkyl, said HET² being optionally substituted with oxo and further optionally substituted with 1-2 substituents independently selected from halo and C₁₋₄alkyl, said C₁₋₄alkyl being optionally substituted with 1-3 halo groups,
   (7) phenoxy or —S(O)₀₋₂phenyl,
   (8) benzyloxy or —S(O)₀₋₂benzyl,
   (9) benzoyl,
   (10) phenyl or naphthyl,
   (11) —O-HET² or —S—HET², said HET² being optionally substituted with oxo and further optionally substituted as defined below, and
   (12) HET³, wherein HET³ is a 5- or 6-membered aromatic or non-aromatic ring, or a benzofused analog thereof, containing from 1 to 4 heteroatoms selected from O, S and N, said HET³ being optionally substituted with oxo and further optionally substituted as defined below,
groups (7)-(12) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups;
R³ is C₁₋₁₀alkyl, optionally substituted with 1-2 oxo or carboxy groups and further optionally substituted with 1-3 substituents independently selected from the group consisting of:
   (a) halo,
   (b) hydroxy
   (c) cyano,
   (d) C₁₋₄alkoxy,
   (e) —NHR⁷, wherein R⁷ is independently H or C₁₋₅alkyl,
   (f) —S(O)₀₋₂C₁₋₄alkyl, and
   (g) HET², wherein HET² represents a 5- to 7-membered aromatic or non-aromatic ring containing 1-4 heteroatoms selected from O, S and NR⁸, wherein R⁸ is independently H or C₁₋₅alkyl, said HET² being optionally substituted with oxo and further optionally substituted with 1-2 substituents independently selected from halo or C₁₋₄alkyl, said C₁₋₄alkyl being optionally substituted with 1-3 halo groups,
each R⁴ is independently selected from the group consisting of: H, halo, hydroxy, C₁₋₆alkyl and C₁₋₄alkoxy, said C₁₋₆alkyl and C₁₋₄alkoxy being optionally substituted with oxo and further optionally substituted with 1-3 halo groups; and
R⁵ is selected from the group consisting of: H, phenyl, naphthyl, C₁₋₆alkyl optionally substituted with OR¹² and 1-3 halo groups, and C₅₋₇ cycloalkyl optionally containing one heteroatom selected from O, S and NR¹³,
wherein R¹² is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy, and
R¹³ is H or C₁₋₄alkyl optionally substituted with 1-3 halo groups; and
R⁶ represents H;
or in the alternative, R⁵ and R⁶ are taken in combination and represent a ring of 4-7 members, said ring optionally containing one heteroatom selected from O, S and NR¹³.

Another embodiment of the invention encompasses a compound of Formula II wherein:
R¹ is selected from the group consisting of:
   (1) halo,
   (2) C₁₋₄alkyl or C₁₋₄alkoxy, each optionally substituted with oxo and 1-3 halo groups, and
   (3) HET³, wherein HET³ is a 5- or 6-membered aromatic or non-aromatic ring, or a benzofused analog thereof, containing from 1 to 4 heteroatoms selected from O, S and N, and optionally substituted with 1-2 substituents independently selected from halo and C₁₋₄alkyl, said C₁₋₄alkyl being optionally substituted with 1-3 halo groups;
R² and each R⁴ are hydrogen;
R⁵ is selected from the group consisting of: C₁₋₆alkyl, phenyl and naphthyl; and
R⁶ is hydrogen.

Within this embodiment HET³ is 1,2,4-oxadiazole, optionally substituted with C₁₋₄alkyl.

The invention also encompasses a method for detecting active caspase-3 in cells or tissues of a mammal comprising contacting said cells or tissues with a compound of Formula I and detecting active caspase-3.

Another embodiment of the invention encompasses a method for detecting active caspase-3 in cells or tissues of a mammal comprising contacting said cells or tissues with a compound of Formula II and detecting active caspase-3.

Another embodiment of the invention encompasses a method for determining the caspase-3 active site occupancy of a sample reversible caspase-3 inhibitor in an animal model of cellular injury comprising:
1) administering to said animal said sample reversible caspase-3 inhibitor;
2) euthanizing said animal and extracting said injured cells;
3) contacting said injured cells ex vivo with a compound of Formula I;
4) detecting the amount of said compound of Formula I to determine the number of caspase-3 free active sites; and
5) comparing said number of caspase-3 free active sites to the total measure of active caspases to determine the caspase-3 active site occupancy.

Another embodiment of the invention encompasses a method for determining the caspase-3 active site occupancy of a sample reversible caspase-3 inhibitor in a cell culture comprising:
1) contacting said cell culture with a sample reversible caspase-3 inhibitor;
2) contacting said cell culture with a compound of Formula I;
3) detecting the amount of said compound of Formula I to determine the number of caspase-3 free active sites; and
4) comparing said number of caspase-3 free active sites to the total measure of active caspases to determine the caspase-3 active site occupancy.

The invention also encompasses a kit for detecting active caspase-3 in cells or tissues of a mammal comprising a compound of Formula I.

Another embodiment of the invention encompasses a kit for detecting active caspase-3 in cells or tissues of a mammal comprising a compound of Formula II.

The invention also encompasses the radiolabeled compound which is 5-fluoro-3-({N-[(5-iodo-2-methoxyphenyl)acetyl]-L-valyl}amino)-4-oxopentanoic acid, or a salt, ester or hydrate thereof.

The invention also encompasses a radiolabeled compound selected from the following table:

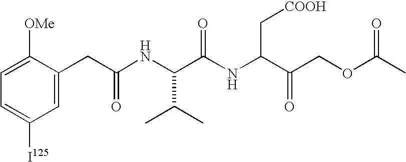

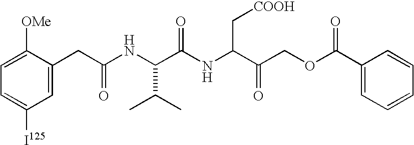

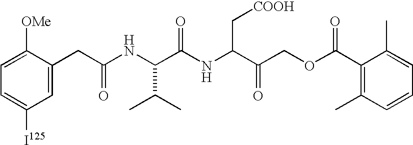

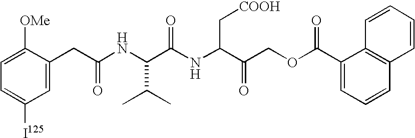

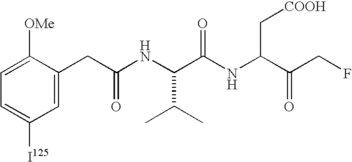

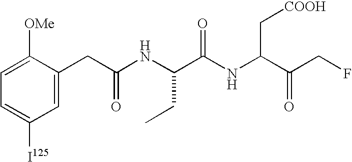

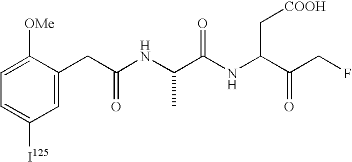

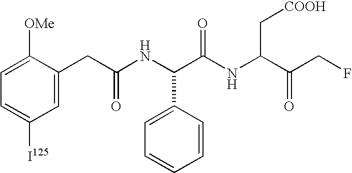

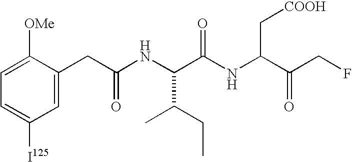

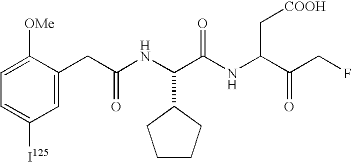

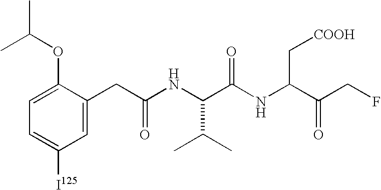

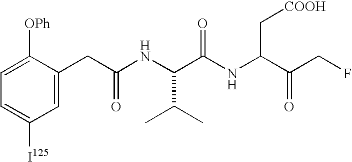

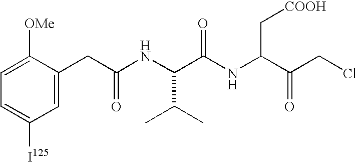

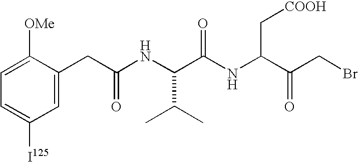

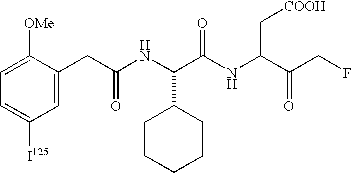

or a salt, ester or hydrate of any of the above.

For purposes of this of this Specification, the phrase “contacting said cells or tissues” means causing the compounds of the present invention to come into contact with the caspase-3 enzyme and bind thereto. In animal models, the phrase includes administering the compounds of the invention to said animals by, for example, i.c.v. injection.

The phrase, “detecting active caspase-3” means detecting caspases-3 by virtue of the labeled compounds bound thereto. Methods for detecting the compounds of the invention are well know in the art and are exemplified in the methods that follow. For example, compounds of the present invention labeled with I¹²⁵ can be detected by autoradiography.

For purposes of this specification alkyl means linear or branched structures and combinations thereof, containing one to twenty carbon atoms unless otherwise specified. Examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl-4-propylnonyl, and the like.

Cycloalkyl means cyclic structures, optionally combined with linear or branched structures, containing one to twenty carbon atoms unless otherwise specified. Examples of cycloalkyl groups include cyclopropyl, cyclopentyl, cycloheptyl, adamantyl, cyclododecylmethyl, 2-ethyl-1-bicyclo[4.4.0]decyl and the like.

Alkoxy means alkoxy groups of one to ten carbon atoms, unless otherwise specified, of a straight, branched or cyclic configuration. Examples of alkoxy groups include methoxy, ethoxy, propoxy, isopropoxy, and the like.

Alkylthio means alkylthio groups of one to ten carbon atoms, unless otherwise specified, of a straight, branched or cyclic configuration. Examples of alkylthio groups include methylthio, propylthio, isopropylthio, etc. By way of illustration, the propylthio group signifies —SCH₂CH₂CH₃.

Halo includes F, Cl, Br and I.

Examples of HET¹ include pyridine, pyrimidine, pyridazine, furan, thiophene, thiazole and oxazole.

Examples of HET² include butyrolactone, tetrahydrofuran, tetrahydropyran, 2-pyrrolidinone, pyridine and pyrimidine.

Examples of HET³ include 1,2,3-oxadiazole, 1,2,4-oxadiazole, 1,3,4-oxadiazole, 1,2,3-thiadiazole, 1,2,4-thiadiazole, 1,3,4-thiadiazole, thiophene, pyrrole, pyridine, tetrazole, oxazole, thiazole, 1,2,3-triazole, 1,2,4-triazole and 1,3,4-triazole.

For purposes of this specification, the following abbreviations have the indicated meanings:
AcOH=acetic acid
Alloc=allyloxycarbonyl
APCI=atmospheric pressure chemical ionization
BOC=t-butyloxycarbonyl
CBZ=carbobenzoxy
DCC=1,3-dicyclohexylcarbodiimide
DIBAL=diisobutyl aluminum hydride
DIFA=N,N-diisoproylethylamine
DMAP=4-(dimethylamino)pyridine
DMF=N,N-dimethylformamide
EDCI=1-(3-dimethylaminopropyl)-3-ethylcarbodiimide hydrochloride
EDTA=ethylenediaminetetraacetic acid, tetrasodium salt hydrate
ESI=electrospray ionization
FAB=fast atom bombardment
FMOC=9-fluorenylmethoxycarbonyl
HMPA=hexamethylphosphoramide
HATU=O-(7-Azabenzotriazol-1-yl)N,N,N′,N′-tetramethyluronium hexafluorophosphate
HOBt=1-hydroxybenzotriazole
HRMS=high resolution mass spectrometry
ICl=iodine monochloride
EBCF=isobutyl chloroformate
KHMDS=potassium hexamethyldisilazane
LDA=lithium diisopropylamide
MCPBA=metachloroperbenzoic acid
Ms=methanesulfonyl=mesyl
MsO=methanesulfonate=mesylate
NBS=N-bromosuccinimide
NMM=4-methylmorpholine
NMO=4-methylmorpholine N-oxide
PCC=pyridinium chlorochromate
PDC=pyridinium dichromate
Ph=phenyl
PPTS=pyridinium p-toluene sulfonate
pTSA=p-toluene sulfonic acid
r.t.=room temperature
rac.=racemic
TFA=trifluoroacetate
TfO=trifluoromethanesulfonate=triflate
TLC=thin layer chromatography
TPAP=tetrapropylammonium perruthenate
   Alkyl group abbreviations:
Me=methyl
Et=ethyl
n-Pr=normal propyl
i-Pr=isopropyl
n-Bu=normal butyl
i-Bu=isobutyl
s-Bu=secondary butyl
t-Bu=tertiary butyl
   L-Amino Acids and Abbreviations:

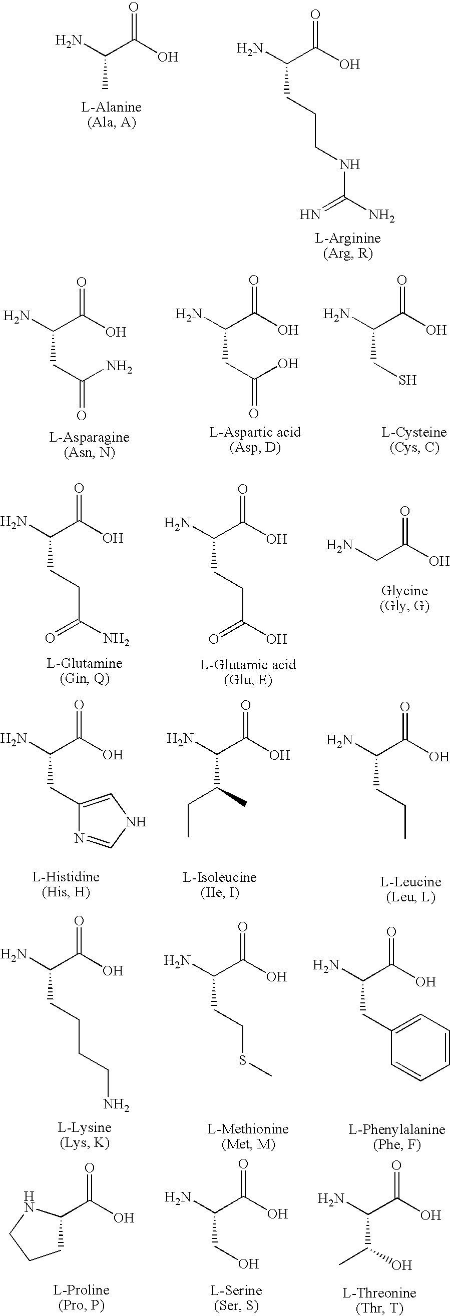

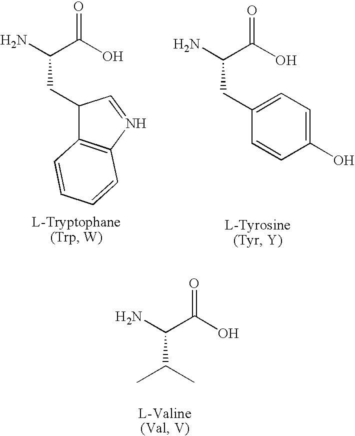

The compounds described herein are intended to include salts, enantiomers, esters, and hydrates, in pure form and as a mixture thereof. Also, when a nitrogen atom appears, it is understood sufficient hydrogen atoms are present to satisfy the valency of the nitrogen atom.

While chiral structures are shown below, by substituting into the synthesis schemes an enantiomer other than the one shown, or by substituting into the schemes a mixture of enantiomers, a different isomer or a racemic mixture can be achieved. Thus, all such isomers and mixtures are included in the present invention.

The compounds described typically contain asymmetric centers and may thus give rise to diastereomers and optical isomers. The present invention is meant to comprehend such possible diastereomers as well as their racemic and resolved, enantiomerically pure forms and pharmaceutically acceptable salts thereof.

Some of the compounds described herein contain olefinic double bonds, and unless specified otherwise, are meant to include both E and Z geometric isomers.

Pharmaceutical compositions may be utilized to administer the compounds of the present invention. Such pharmaceutical compositions comprise a compound of Formula I or Formula II in combination with a pharmaceutically acceptable carrier, and optionally other therapeutic ingredients. The term “salts” refers to salts prepared from pharmaceutically acceptable bases including inorganic bases and organic bases. Representative salts derived from inorganic bases include aluminum, ammonium, calcium, copper, ferric, ferrous, lithium, magnesium, manganic salts, manganous, ammonium, potassium, sodium, zinc and the like. Particularly preferred are the calcium, magnesium, potassium, and sodium salts. Representative salts derived from pharmaceutically acceptable organic bases include salts of primary, secondary and tertiary amines, substituted amines including naturally occurring substituted amines, cyclic amines, and basic ion exchange resins, such as arginine, betaine, caffeine, choline, NN′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Examples of such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric and tartaric acids.

The magnitude of dose of a compound of Formula I or Formula II will, of course, vary with the model being tested and with the particular compound of Formula I. Representative amounts of the compounds for use in the present invention are exemplified in the examples that follow. The appropriate amount of the compound to be used

Any suitable route of administration may be employed for providing a dosage of a compound of the present invention. For example, oral, parenteral and topical may be employed. Dosage forms include tablets, troches, dispersions, suspensions, solutions, capsules, creams, ointments, aerosols, and the like.

The compositions include compositions suitable for oral, parenteral and ocular (ophthalmic). They may be conveniently presented in unit dosage form and prepared by any of the methods well-known in the art of pharmacy.

In practical use, the compounds of Formula I of Formula II may be combined as the active ingredient in intimate admixture with a pharmaceutical carrier according to conventional pharmaceutical compounding techniques. The carrier may take a wide variety of forms depending on the form of preparation desired for administration. In preparing the compositions for oral dosage form, any of the usual pharmaceutical media may be employed, such as, for example, water, alcohols, oils, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case or oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

Pharmaceutical compositions of the present invention suitable for oral administration may be presented as discrete units such as capsules, cachets or tablets each containing a predetermined amound of the active ingredient, as a powder or granules or as a solution or a suspension in an aqueous liquid, a non-aqueous liquid, an oil-in-water emulsion or a water-in-oil emulsion. Such compositions may be prepared by any of the methods of pharmacy but all methods include the step of bringing into active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Methods of Sythesis

Example 1 of the present invention can be synthesized according to the following synthetic scheme:

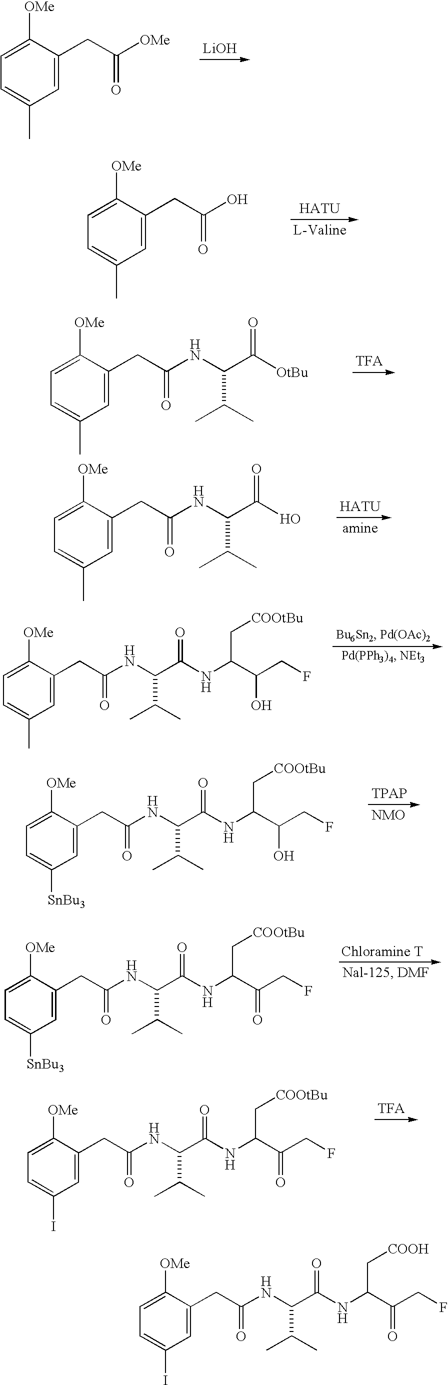

Compounds of the invention can be made by one having ordinary skill in the art in an analogous manner to the above scheme.

### REPRESENTATIVE EXAMPLES

Exemplifying the invention are the following compounds:

### Example 1

### 5-fluoro-3-({N-[(5-iodo-2-methoxyphenyl)acetyl]-L-valyl}amino)-4 oxopentanoic acid (29)

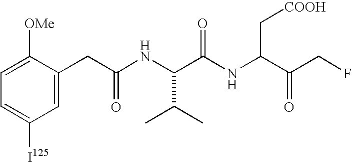

### Step 1: (5-iodo-2-methoxyphenyl)acetic acid (21)

To a solution of methyl (5-iodo-2-methoxyphenyl)acetate (0.92 g, 3 mmol) in 20 mL of 2:1:1 THF:MeOH:water was added lithium hydroxide (8 mmol) and the solution was stirred for two hours. The reaction was then quenched with 1N HCl (8 mL of a 1M aqueous solution, 8 mmol) and then extracted with EtOAc. The organic phases were then combined, dried over MgSO₄, filtered and concentrated in vacuo. The compound was purified by flash chromatography using 40-100% EtOAc/hexanes to yield 0.9 g of 21 as a white solid. ¹H NMR (400 MHz, acetone-d₆): δ 10.9 (br s, 1H), 7.6 (s, 2H), 6.8 (m, 1H), 3.8 (s, 3H), 3.6 (s, 21).

### Step 2: N-[(1S)-1-acetyl-2-methylpropyl]-2-(5-iodo-2-methoxyphenyl)acetamide (22)

To a solution of 21 (0.9 g, 3 mmol) in 50 mL CH₂Cl₂ was added L-valine tert-butyl ester hydrochloride (0.84 g, 4 mmol), EDCI (0.86 g, 4.5 mmol) and diisopropylethylamine (1.6 mL, 9.0 mmol) sequentially followed by stirring for 3 hours. The reaction was then quenched with 1N HCl (10 mL of a 1M aqueous solution, 10 mmol) and then extracted with EtOAc. The combined extracts were then washed with saturated NaRCO₃ followed by brine. The organic phases were then combined, dried over MgSO₄, filtered and concentrated in vacuo. The compound was purified by flash chromatography using 10-50% EtOAc/hexanes to yield 1.0 g of 22 as a colorless oil. ¹H NMR (400 M, acetone-d₆): δ 7.5 (m, 2H), 7.0 (bd, 1H), 6.8 (d, 1H), 4.3 (dd, 1H), 3.8 (s, 3H), 3.5 (AB quartet, 2H), 2.1 (m, 1H), 1.4 (s, 9H), 0.95 (d, 3H), 0.9 (d, 3H).

### Step 3: N-[(5-iodo-2-methoxyphenyl)acetyl]-L-valine (23)

To a solution of 22 (1.0 g, 2.2 mmol) in 10 mL CH₂Cl₂ was added 10 mL of TFA. The solution was stirred for 2 hours and then concentrated in vacuo. Ether was added to the crude oil until the product precipitated. The organic layer was decanted and the solid was pumped dry to yield 0.9 g of 23 as a white solid. ¹H NMR (400 MD, acetone-d₆): δ 7.5 (m, 2H), 6.8 (d, 1H), 4.5 (dd, 1H), 3.8 (s, 3H), 3.5 (AB quartet, 2H), 2.2 (m, 1H), 0.95 (d, 3H), 0.9 (d, 3H).

### Step 4: N¹-(1-ethyl-3-fluoro-2-hydroxypropyl)-N²-[(5-iodo-2-methoxyphenyl)acetyl]-L-valinamide (25)

To a solution of 24 (391 mg, 1.0 mmol) in 5 mL DMF was added the tert-butyl 3-amino-2,3,5-trideoxy-5-fluoropentonate (prepared via the known sequence in Tetrahedron Letters 1994, 35.52, 9693), (170 mg, 0.82 mmol), HATU (380 mg, 1 mmol) and diisopropylethylamine (1.6 mL, 9.0 mmol) and the solution was stirred for 3 hours. Water was added and the solution was extracted with EtOAc. The organic phases were then combined, dried over MgSO₄, filtered and concentrated in vacuo. The compound was purified by flash chromatography using 10-50% EtOAc/hexanes to yield 250 mg of 25 as a colorless oil as a mixture of four diastereomers. ¹H NMR (400 MHz, acetone-dr): δ 7.5 (d, 2H), 7.4-6.9 (d, 1H), 6.8 (m, 1H), 4.84.65 (d, 1H), 4.64.1 (m, 3H), 3.8 (s, 3H), 3.5 (AB quartet, 2H), 2.7-2.4 (m, 2H), 2.1 (m, 1H), 1.4 (s, 9H), 0.95 (m, 6H).

### Step 5: N¹-(1-ethyl-3-fluoro-2-hydroxypropyl)-N²-{[2-methoxy-5-(tributylstannyl)phenyl]acetyl}-L-valinamide (26)

To a solution of 25 (116 mg, 0.2 mmol) in 5 mL triethylamine was added Pd(OAc)₂ (10 mg, 0.044 mmol), Pd(PPh₃)₄ (25 mg, 0.022 mmol) at 50° C. was added hexabutylditin (0.2 mL, 0.4 mmol) and the resulting solution was heated to 100° C. and stirred for two hours. The solution was then cooled and filtered through a pad of celite with ether and concentrated in vacuo. The compound was purified by flash chromatography using 10-50% EtOAc/hexanes to yield 70 mg of 26 as a colorless oil. ¹H NMR (400 MHz, acetone-d₆): δ 7.5 (d, 1H), 7.3 (m, 2H), 7.0 (m, 1H), 6.8 (m, 1H), 4.74.6 (d, 1H), 4.54.1 (m, 3H), 3.8 (s, 3H), 3.5 (AB quartet, 2H), 2.7-2.4 (m, 2H), 2.1 (m, 1H), 1.5 (m, 18H), 1.4 (s, 9H), 1.0-0.8 (m, 9H), 0.95 (m, 6H).

### Step 6: N¹-(1-ethyl-3-fluoro-2-oxopropyl)-N²-{[2-methoxy-5-(tributylstannyl)phenyl]acetyl}-L-valinamide (27)

To a solution of 26 (10 mg) in 2 mL CH₂Cl₂ was added NMO (20 mg) followed by a crystal of TPAP and the resulting solution was stirred for 1 hour. The solution was concentrated in vacuo and the compound was purified by flash chromatography using 50% ethyl acetatethexanes to yield 10 mg of 27 as a colorless oil. ¹H NMR (400 MB, acetone-d₆): δ 7.8-7.7 (d, 1H), 7.3 (m, 2H), 7.2 (m, 1H), 7.0 (m, 1H), 5.3-5.0 (m, 2H), 4.84.7 (m, 1H), 4.2 (m, 1H), 3.8 (s, 3H), 3.5 (AB quartet, 2H), 2.9-2.7 (m, 2H), 1.6-0.8 (m, 42H).

### Step 7: N¹-(1-ethyl-3-fluoro-2-oxopropyl)-N²-[(5-iodo-2-methoxyphenyl)acetyl]-L-valinamide (28)

To a solution of 27 (2 mg) in 0.2 mL DMF was added sodium iodide (5 mCi dissolved in 0.1 mL water) followed by chloramine T (2 mg dissolved in 0.2 mL 1:1 DMF:water). The resulting solution was stirred for two hours and then purified directly by prep HPLC. Data shown is for the non-radioactive compound. ¹H NMR (400 MHz, CDCl₃): δ 8.1-7.7 (d, 1H), 7.5 (m, 2H), 7.2 (d, 1H), 6.8 (d, 1H), 5.3-5.0 (m, 2H), 4.8-4.6 (m, 1H), 4.2 (m, 1H), 3.8 (s, 3H), 3.5 (m, 2H), 2.9-2.6 (m, 2H), 2.10 (m, 1H), 1.4 (s, 9H), 0.90 (m, 6H).

### Step 8: 5-fluoro-3-({N-[(5-iodo-2-methoxyphenyl)acetyl]-L-valyl}amino)-4-oxopentanoic acid (29)

To a solution of 28 (from the previous experiment) in 5 mL CH₂Cl₂ was added 2 mL of TFA. The solution was stirred for 2 hours and then concentrated in vacuo to yield 29. Data shown is for the non-radioactive compound. ¹H NMR (400 MHz, CDCl₃): δ 8.1-7.7 (d, 1H), 7.5 (m, 2H), 7.2 (d, 1H), 6.8 (d, 1H), 5.3-5.0 (m, 2H), 4.8-4.6 (m, 1H), 4.2 (m, 1H), 3.8 (s, 3H), 3.5 (m, 2H), 2.9-2.6 (m, 2H), 2.10 (m, 1H), 0.90 (m, 6H).

The following examples can by synthesized by following the methods of synthesis described above and procedures analogous to Example 1:

| Ex. | Structure |
|---|---|
| 2 | 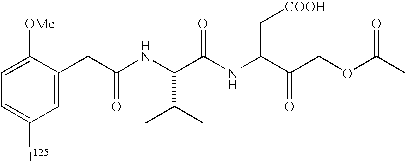 |
| 3 | 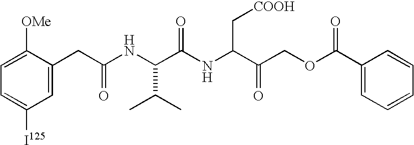 |
| 4 | 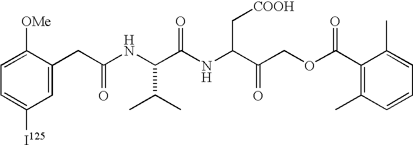 |
| 5 | 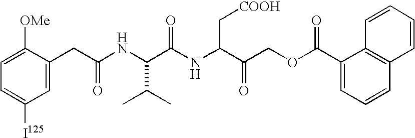 |
| 6 | 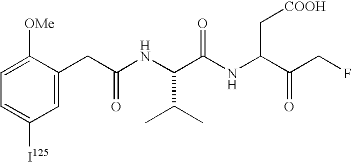 |
| 7 | 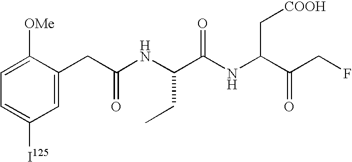 |
| 8 | 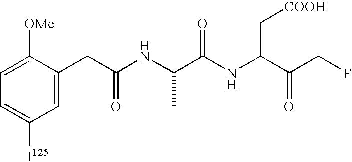 |
| 9 | 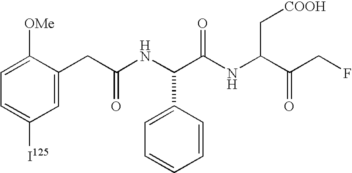 |
| 10 | 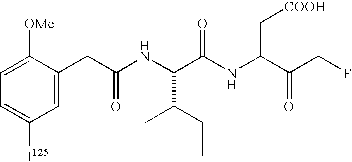 |
| 11 | 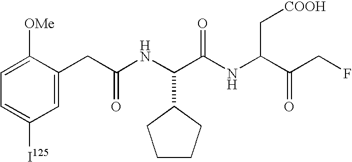 |
| 12 | 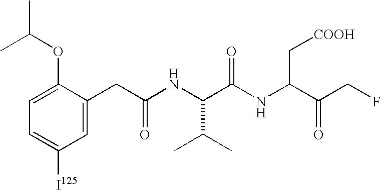 |
| 13 | 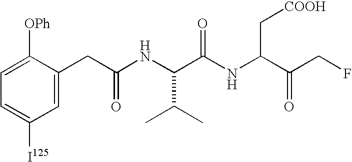 |
| 14 | 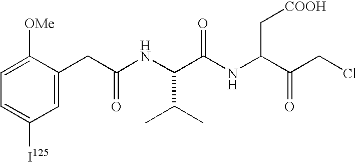 |
| 15 | 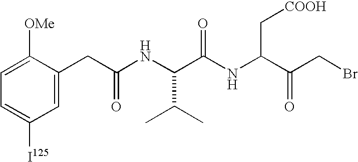 |
| 16 | 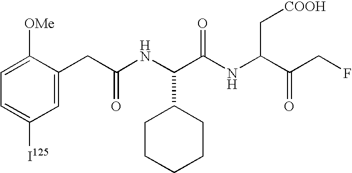 |

Utility

The compounds of the present invention are useful as caspase active site probes in a variety of in vitro or in vivo assays or models. For example, compounds of the present invention would be useful as follows:
in vitro-labeling of caspases (purified and from tissue protein extracts from animal models of cellular injury), for example, Hypoxia-ichemia in neonatal rats, Cecal-ligation and puncture in rat and mouse, LCAO model of myocardial infarct in the rat, mouse or rabbit.
Determination of caspase active site occupancy by reversible caspase inhibitor in vitro.
Labeling of active caspases in cells and in blood, for example, thymocytes, NT2, Jurkats, rat white blood cells.
Determination of caspase active site occupancy by reversible caspase inhibitors in cell culture, for example, thymocytes, Jurkats, NT2 cells.
Caspase labeling in vivo in animal models of cellular injury, for example, Hypoxia-Ischemia in neonatal rats, Cecal-ligation and puncture-induced peritonitis in mouse.
Determination of caspase active site occupancy following animal dosing with caspase inhibitor ex-vivo, for example, CLP rats, ex vivo labeling performed on thymocytes.
   Methods

Unless otherwise indicated, the specific activity of the compound of the present invention used in the methods that follow is 2000 Ci/mmole.

For purposes of this Specification, the designation M791 means the compound (3S)-5-(benzylthio)-3-[(N-{[2-methoxy-5-(3-methyl-1,2,4-oxadiazol-5-yl)phenyl]acetyl}-L-valyl)amino]4-oxopentanoic acid, which has the following structure: 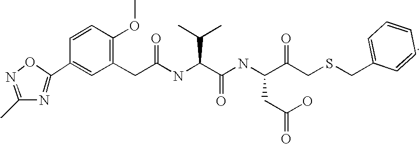
Methods for making this compound are disclosed in U.S. Pat. No. 6,225,288, granted May 1, 2001.

For purposes of this Specification, the designation M867 means the compound (3S)-3-({(2S)-2-[5-tert-butyl-3-{[(4-methyl-1,2,5-oxadiazol-3-yl)methyl]amino}-2-oxopyrazin-1(2H)-yl]butanoyl}amino)-5-[methyl(pentyl)amino]-4-oxopentanoic acid, which has the following structure: 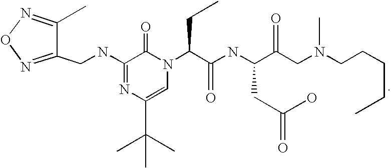
Methods for making this compound are disclosed in U.S. Pat. No. 6,444,811 granted Sep. 3, 2002.

For purposes of this Specification, the designation [¹²⁵I]-M808 means the radiolabeled caspase inhibitor of Example 1. The designation M808 means the corresponding unlabeled caspase inhibitor of Example 1.

For purposes of this Specification, MF R280 is an anti-human caspase-3 antibody and MF467 is an anti-rat caspase-3 antibody. MF R280 is described in Roy et al (2001) PNAS 98:6132-6137.

Ac-DEVD-AMC (AMC, amino-4-methylcoumarin) has the following structure: 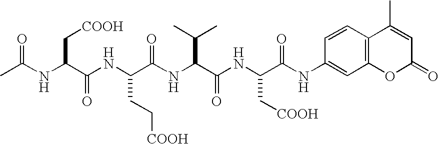
This compound may be prepared as follows: i) synthesis of N-Ac-Asp(OBn)-Glu(OBn)-Val-CO₂H, ii) coupling with Asp(OBn)-7-amino-4-methylcoumarin, iii) removal of benzyl groups.
Tissue, Cell Extracts

Tissues (rat liver, kidney, brain, thymus, mouse thymus) were either frozen or processed within 20 minutes of their removal. Rat tissues and cultured cells were homogenized in ice cold cell lysis buffer (50 mM Tris-Cl pH 7.5, 2 mM EDTA, 1% NP-40) supplemented with 5 mM DTT and Complete protease inhibitor cocktail (Roche). Thymi from mice injected with [¹²⁵I]-M808 were homogenized in lysis buffer supplemented with 25 μM of M808. Nuclear debris was removed by centrifugation at 13000 rpm for 10 min and the soluble fraction quantitated by Bradford assay (Bio-Rad). Caspase-3 like activity was determined with the fluorogenic substrate Ac-DEVD-AMC as described by Roy et al. (2001) Proc. Nat. Acad. Sci. USA 98: 6132-6137.

Thymocyte Cell Suspension

Rat thymocytes were processed within 20 min of their removal. A single-cell suspension was obtained by grinding the thymus in a 50 μM Medicon (Dako) and Medimachine (Dako) with 2 ml of ice-cold thymocyte isolation buffer (PBS, 2 mM glucose, 2 mM L-glutamine, 1% FBS), with 2×15 second pulse. The cell suspension was filtered through a 50 μM nylon mesh (Becton-Dickinson). Cells were centrifuged at 300 g for 10 min at 4° C., suspended in 10 ml of hypotonic lysis buffer (17 mM Tris-Cl pH 7.5; 140 mM NH₄Cl) and incubated for 10 min at room temperature. Thymocytes were pelleted by centrifugation at 300 g for 10 min, and suspended in 5-10 ml of thymocyte isolation buffer without FBS. Cells were counted with hemocytometer and trypan blue staining.

Cell Culture

Jurkat cells (ATCC # TEB-152) were maintained in RPMI1640 supplemented with 10% FBS, 2 mM L-glutamine and antibiotics (penicillin and streptamycin). Apoptosis was induced on cells (1 million cells/ml) that had been starved for 36 hours in CytoSf4 media (Kemp technologies) by addition of camptothecin (Sigma; 3 μg/ml). NT2 cells (ATCC 3813555) were cultured in DMEM+10% FBS+2 mM L-glutamine and antibiotics. Apoptosis induced with camptothecin (5 μg/ml) in culture media.

Rat thymocytes were cultured for 24 hours at a density of 10×10⁶ cells/ml in CytoSF4 supplemented with 2 mM L-glutamine and penicillin-streptamycin and the indicated concentration of caspase inhibitors.

Animals and Surgical Procedures

Female Sprague-Dawley rats (250-300 g; Charles River, St-Constant, Qc, Canada) and ND4 mice (20-25 g Harlane Sprague-Dawley) were housed in a 12 hour light/dark cycle with free access to food and water. All procedures were carried out under appropriate Animal Care Committee approval in strict accordance to Merck and Co. animal care policies. For Cecal Ligation and Perforation, animals were anesthetized with 2.5% isoflurane and body temperature maintained by use of a thermoregulated heated blanket. A midline incision was made in the abdominal wall of the animal and the cecum exteriorized. The cecum was ligated with a nylon (4-0) suture proximal to the ileocecal valve. In mice, perforation of the cecum was done using a 23 g needle passed through the distal portion of the cecum. Femoral vein cannulation of rats was performed by a small incision in the inguinal region and the femoral vein was isolated. A Silicone catheter (0.02″×0.037″, Lomir) connected to a polyurethane catheter (PU-C30, 3 French, 80 cm, Instech Solomon) was inserted into the vena cava, exteriorized at the nape of the neck and clamped for the duration of the surgery. Cannulation was immediately followed by cecal ligation and perforation, using a 20 g cannula (Abbott Ireland, Sligo. Rep. of Ireland) passed through-and-through the distal portion of the exteriorized cecum. Braided silk (size 0) was threaded through the cannula and secured in place to allow leakage of the cecal content into the peritoneum. Sham-operated animals had the cecum exteriorized but no ligation or puncture of the cecum. The abdominal wall was sutured with PDS (4-0) and the skin sutured with surgical glue (mice) or clips (rats). Immediately after surgery, all animals received 1 c.c. of 0.9% saline administered by subcutaneous injection. A bolus of vehicle or compound (M867) was administered via the intravenous catheter, which was then connected to a Medfusion 2010i Syringe pump (Medex Inc. Duluth, Ga., USA) at delivery rate 2 ml/hr/kg for 24 hours.

Apoptosis Detection

DNA fragmentation was determined by flow cytometry on 1.5×10⁶ thymocytes or 500,000 Jurkat cells fixed for 30 min on ice in 80% EtOH. The cells were pelleted by centrifugation at 300 g for 10 min, suspended in PBS and incubated on ice for 45 min. Pelleted cells were suspended in 500 μl of PI staining solution (PBS/0.1% tritonX-100/RNAse A (Roche)/25 μg/ml propidium iodide (PI; Sigma). Flow cytometry was performed with a FACSCalibur instrument (Becton-Dickinson) on 20,000 events/sample, each sample prepared in duplicate. Very small cell debris was electronically gated out based on forward light scatter. NT2 cell DNA fragmentation was quantitated using the Cell Death Detection ELISA kit (Roche) according to the manufacturer's specifications. All assays were performed on protein extract that had not been frozen as we have found that freeze-thawing protein extract alters reactivity. Several dilutions were prepared for each sample to ensure that signal would be found within the assays linear range.

[¹²⁵I]-M808 Labeling

The determination of caspase activity by [¹²⁵I]-M808 labeling was performed by pre-incubating purified recombinant human caspase-3 with indicated amounts of caspase inhibitor (M791) or vehicle (DMSO), for 1 hour at 25° C. in ICE buffer III (50 mM Hepes-KOH pH 7.0, 0.1% CHAPS, 10% sucrose, 2 mM EDTA), supplemented with 5 mM DTT. [¹²⁵I]-M808 was added to a final concentration of 1.25 μM and incubated for 5 min at 25° C. Active caspases in tissue or cell extracts were labeled by incubating 60-80 μg of lysate with [¹²⁵I]-M808 for 15 minutes at 25° C. Labeling was stopped by addition of Laemli buffer (2% SDS; 0.35 M O-mercaptoethanol; 50 mM Tris-Cl pH 6.8; 10% glycerol; 0.05% bromophenol blue) and by heating at 95° C. for 5 minutes. Caspase labeling in whole cells was performed as following. NT2 cells were plated in 24-well dishes (50 000 cells/well) in DMEM+10% FBS. The next day, media was replaced by media containing camptothecin (5 μg/ml) and the indicated amounts of caspase inhibitors. The culture media was removed after 5 hours of incubation and replaced by 100 μl of fresh media containing 12.5 nM [¹²⁵]-M808. After a 1 h incubation at 37° C., the media was removed, the cells were washed once with PBS and lysed in lysis buffer+25 μM M808. Cellular debris was removed by centrifugation at 13000 rpm for 10 min and boiled at 95° C. in Laemli buffer. Apoptotic Jurkat cells and rat thymocytes were labeled with [¹²⁵I]-M808 in essentially the same fashion. Briefly, starved Jurkat cells (0.5 million) plated in CytoSF4 with camptothecin (5 μM/ml) and the indicated concentration of caspase inhibitors for 5 hours. The cells were then pelleted by centrifugation at 350 g for 10 min and the cell pellets were suspended in CytoSF4 media containing 12.5 nM [¹²⁵]-M808 and the indicated concentration of caspase inhibitor, for a one hour incubation period at 37° C. Labeling was stopped by washing the cells with 1 ml of PBS+25 μM M808. The cells were pelleted by centrifugation and lysed as described. Rat thymocytes were treated the same way except that 3 million cells/assay were used, and that apoptosis occurred spontaneously over a 24 h culture period in CytoSF4 media. Labeling with 12.5 nM [¹²⁵I]-M808 was performed for 15 minutes at 37° C. In vivo labeling of active caspases was performed the intravenous injection of 125 μl of a 0.43 μM solution of [¹²⁵I]-M808 (1125 Ci/mmole) in 10% PEG200. Assuming an average blood volume of 1.25 ml per mouse, the expected initial blood concentration of [¹²⁵I]-M808 is 43 nM. Animals were euthanized 45 minutes after [¹²⁵I]-M808 injection. Thymi were recovered and processed for protein extraction as described above.

Electrophoresis and Western Blotting

Denatured protein extracts in 1× Laemli buffer were resolved through a 18% acrylamide gradient SDS-PAGE gel (Invitrogen) at 30 mA in Tris-Glycine buffer. Samples labeled with [¹²⁵I]-M808 were migrated until the dye front reached 0.5 cm above the end of the gel, and acrylamide 1 cm above the dye front was cut out to remove all free [¹²⁵I]-M808 and reduce background. The gels were fixed for 45 minutes in a 40% Methanol-10% Acetic acid solution and dried for 90 min at 80° C. before exposure on MS Kodak film with MS intensifying screens. For Western blotting, proteins were transferred onto a 45 μm nitrocellulose membrane (Invitrogen) in Tris-Glycine-20% methanol buffer at 40V for 2 hours. Non-specific protein binding was minimized by incubating the membranes for 1 hour in blocking buffer (5% non-fat milk/TBS/0.1% Tween-20). Anti-caspase-3 (W R280 and MF467) were diluted 2000-fold in blocking buffer and incubated for 1 hour at 25° C. Membranes were washed in TBS/0.1% Tween-20 and incubated for 45 min with horseradish-peroxidase-coupled anti-rabbit IgG antibody (Amersham/Pharmacia) diluted 5000-fold in blocking buffer. Chemiluminescence reaction was performed with Supersignal West Femto chemiluminescent reagent (Pierce) and exposed to Hyperfilm ECL (Amersham/Pharmacia). Densitometry was performed using a BioRad instrument and QuantityOne software. Statistical analysis performed by ANOVA.

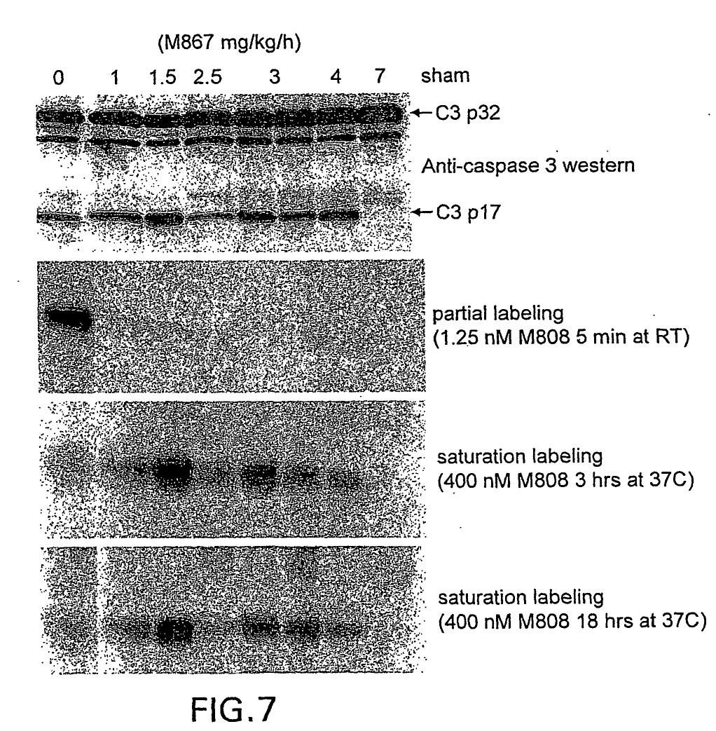

Saturation Labeling of Caspases in Protein Extracts (FIG. 7)

Quantitation of total amount of active caspase can be determined by exposing protein extracts from either cultured cells (Jurkats, thymocytes) of CLP-derived rat thymi to low-specific activity [¹²⁵I]-M808 for long period of time. Briefly, 80 μg of rat thymus protein extracts from CLP-operated rats dosed with M867, were incubated for 3 hours or 18 hours with 1250 M808 (5 nM at 2000 Ci/mmole) and M808 (395 nM). In parallel, a short incubation (5 min) on 80 μg of extract was also performed high specific activity [¹²⁵I]-M808 (1.25 nM at 2000 Ci/mmole). The labeling reaction was stopped by the addition of Laemli buffer and boiling, and proteins were resolved by SDS-PAGE. No change in [¹²⁵I]-M808 signal intensity was noted between 3 and 18 hours of labeling. No change in signal rations between samples was noted. A good correlation between [¹²⁵I]-M808 signal intensity and caspase-3 p17 band was established, indicating that 3 hours is sufficient to label all active caspase-3 quantitatively.

Results

Active site shared between large and small caspase subunit, with active site cysteine located on the large subunit. Covalent bond expected to form on p17 caspase subunit.

M808 Specificity and Sensitivity In Vitro

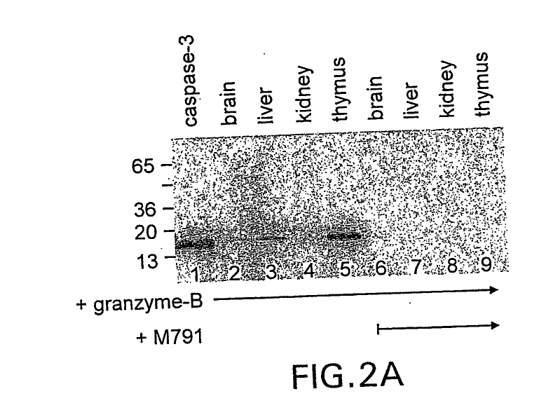

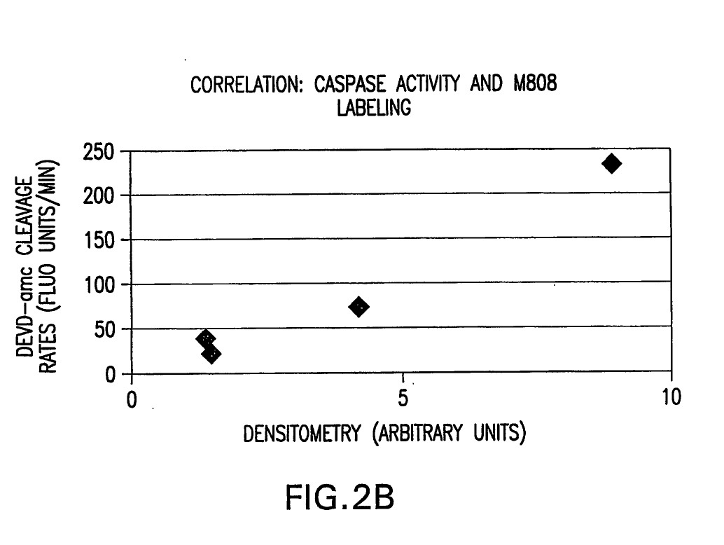

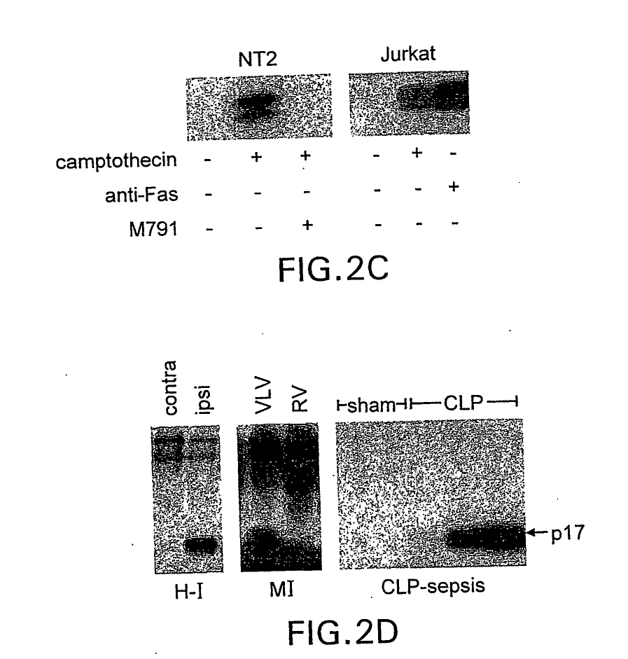

We first tested the specificity of [¹²⁵]-M808 for active caspases in crude protein extracts. Equal amounts various rat tissues homogenates were either incubated with [¹²⁵I]-M808 or pretreated with granzyme-B prior to the addition of M808. In parallel, caspase activity in granzyme B treated extracts was measured by DEVD-AMC cleavage. As expected, the p17 subunit of purified recombinant human caspase 3 covalently bound [¹²⁵I]-M808 (FIG. 2A lane 1). All granzyme-B treated rat tissues contained a [¹²⁵I]-M808-lableled p19 band, while liver and thymus exhibited an additional band migrating at 20 kDa (lanes 2-5). The identity of the radiolabeled proteins as caspases was ascertained by several means. No radiolabeled proteins migrating at this position was observed when granzyme-B treatment was omitted (data not shown). No radiolabeling (lanes 6-8) or strongly reduced labeling (lane 9) was seen if the caspase-3 specific inhibitor M791 was added to the extract prior to [¹²⁵I]-M808. A very good correlation between the total DEVDase activity and densitometry of the p19/p20 labeled protein was established (FIG. 2B). In extracts from cultured cells treated with the apoptotic inducer camptothecin or anti-Fas (FIG. 2C), [¹²⁵I]-M808 recognized polypeptides migrating at 17, 19 and 20 kda. These bands were absent if the apoptotic inducer was omitted, or if M791 was added. Taken together, these results demonstrate that M808 specifically recognizes caspases, including caspase-3, in a complex protein mixture.

Using purified recombinant human caspase-3, the sensitivity threshold of [¹²⁵I] M808 was established at 0.5 fmoles (0.02 ng; data not shown). We determined whether [¹²⁵I]-M808 would be sufficiently sensitive to detect active caspases in animal model of diseases where it is believed apoptosis contributes to the pathology. Extracts from hypoxic-ischemic (HI) neonatal rat brain, infarcted rat heart, cortex and hippocampus, and septic rat thymi, were incubated with [¹²⁵I]-M808 and resolved by SDS-PAGE (FIG. 2D). A weak radiolabeled signal migrating at 17 kDa was observed in ischemia-injured tissue, but not in non-injured contralateral tissue from HI rat brain and infarcted rat hearts. Strong labeling was observed in septic but not sham-operated rat thymi, with an M791-competable signal. No specific signal was observed in ischemic adult rat cortex and hippocampi (data not shown). Thus, [¹²⁵]-M808 specifically recognizes active caspases in several types of injured tissues.

[¹²⁵I]-M808 Measures Caspase Activity In Vitro

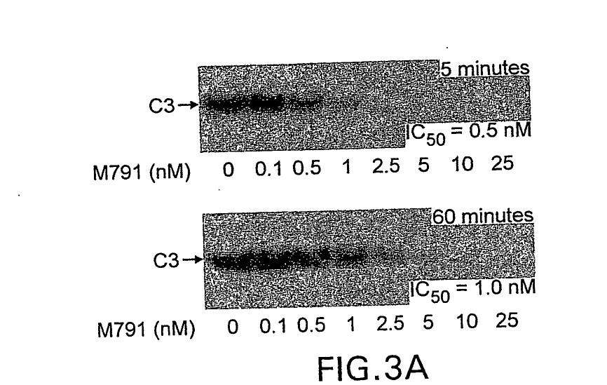

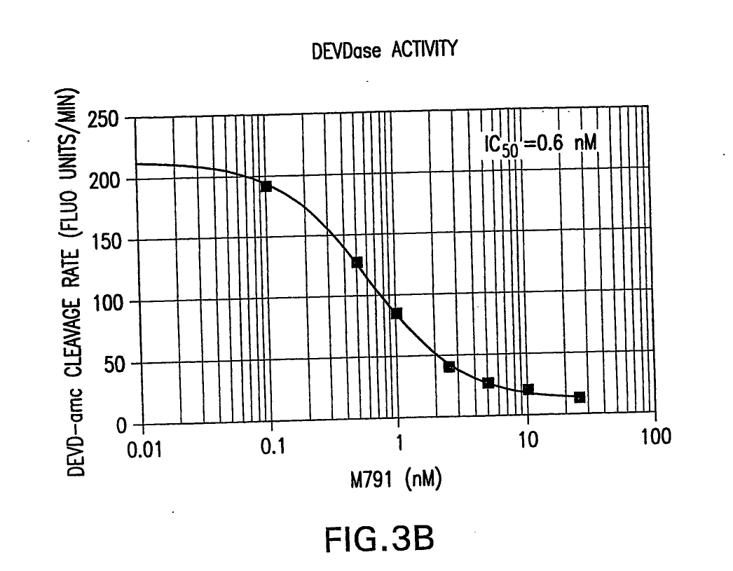

We have shown that [¹²⁵I]-M808 is both a sensitive and specific probe that recognized active caspases in cultured cells treated with apoptotic inducers or in injured tissues. A potential use of [¹²⁵I]-M808 as an active site probe is the determination of the amount of caspase activity that remains in cells treated with reversible caspase inhibitors. The ability of [¹²⁵]-M808 to accurately measure the amount of active caspases was first verified using purified recombinant caspase-3 that had been pre-incubated with increasing amounts of M791 (FIG. 3). One half of the mixture was used for DEVDase activity while [¹²⁵I]-M808 was added to the other portion for 5 minutes before resolving on SDS-PAGE. IC₅₀ was measured at 0.6 nM by DEVD cleavage and at 0.45 nM. Incubating with [¹²⁵I]-M808 for 60 minutes instead of 5 increased the apparent IC₅₀ to 1 nM. Thus, the use of [¹²⁵I]-M808 conjugated to SDS-PAGE reflect the amount of active caspase present in a mixture of enzyme and reversible inhibitor. An increase in the apparent IC₅₀ is observed if the probe is left for longer periods of time, most likely owed to the dissociation of the reversible M791 and its replacement by the irreversible [¹²⁵I]-M808.

[¹²⁵I]-M808 Measures Caspase Active Site Occupancy in Cells

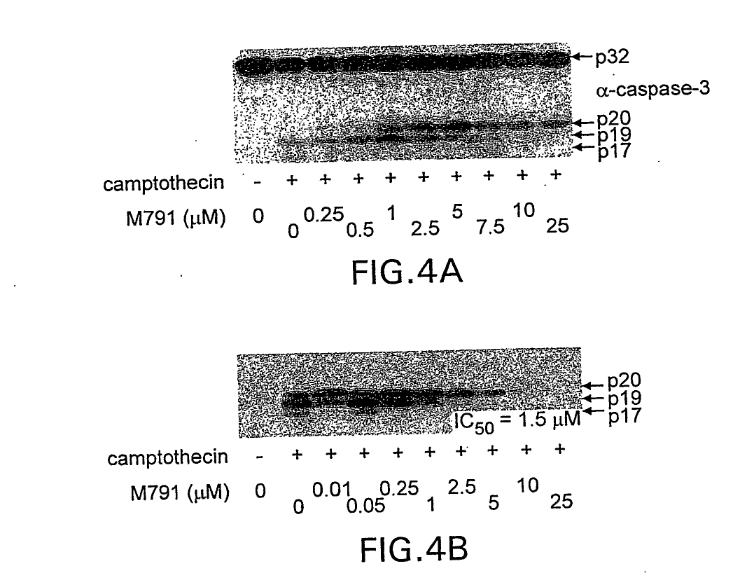

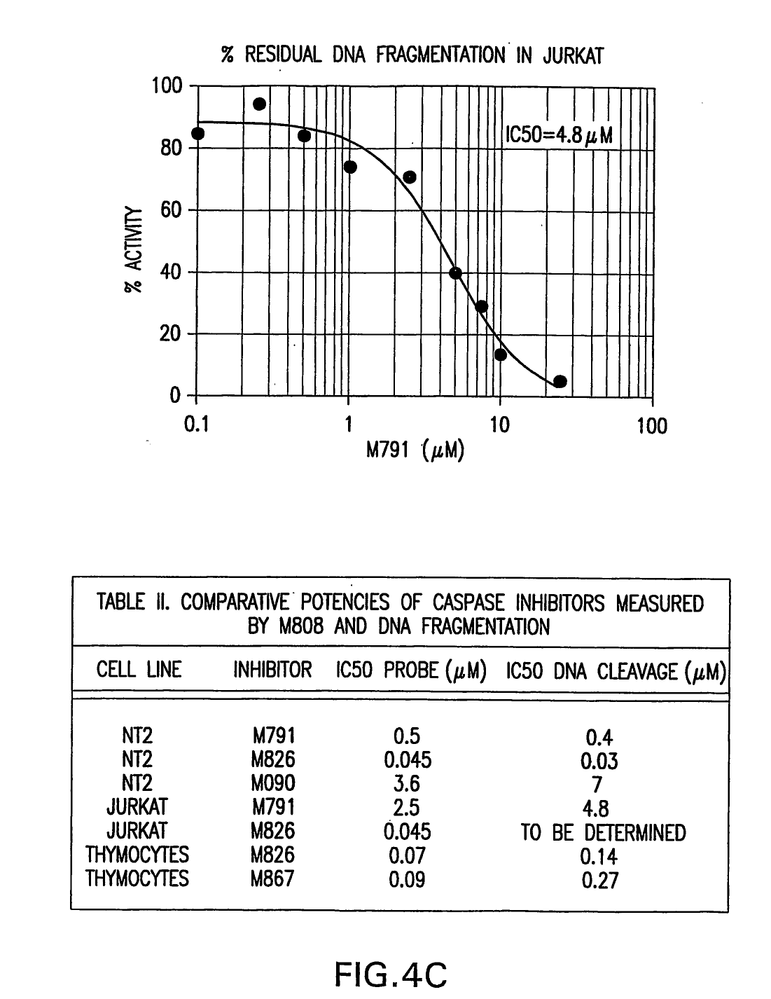

We then assessed whether [¹²⁵I]-M808 could detect active caspase in whole cells and reflect residual caspase activity in the presence of reversible caspase inhibitors. Jurkat cells were induced to undergo apoptosis for 5 hours. One hour prior to the termination of the incubation period, [¹²⁵]-M808 was added in the culture media. An anti-caspase-3 western blot in which cells were radiolabeling was omitted, reveals the presence of varying amounts of p17, p19 and p20 form of processed caspase 3 in all camptothecin-treated lanes (FIG. 4). Although M791 prevents the auto-processing of p20 caspase 3 into p19 and p17, it does not block the overall formation of p20. Addition of [¹²⁵I]-M808 in the culture media resulted in the labeling of all 3 forms of caspase-3. M867 occupancy of active caspase-3 was evident from the fact that the intensity of the radiolabeled bands decreased as the concentration of M867 increased (FIG. 4).

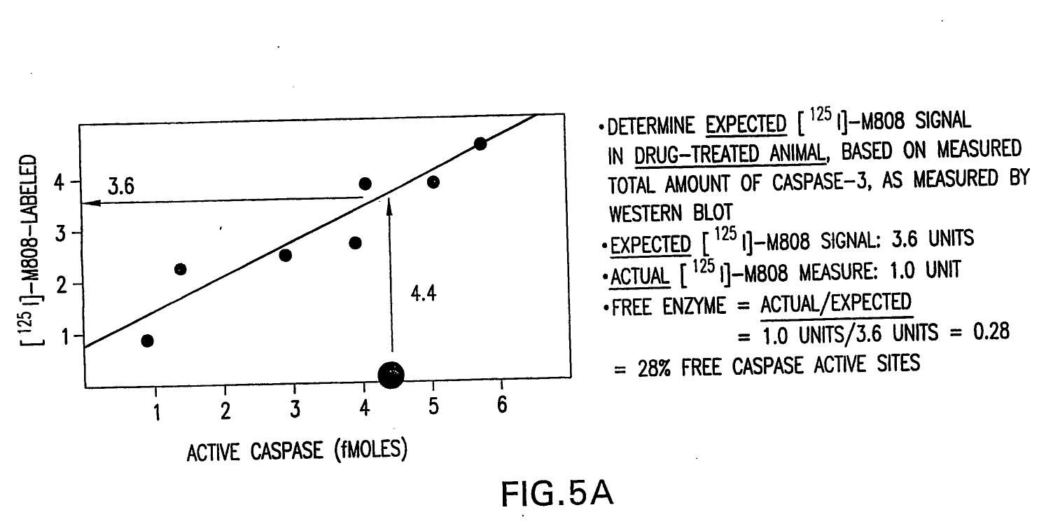

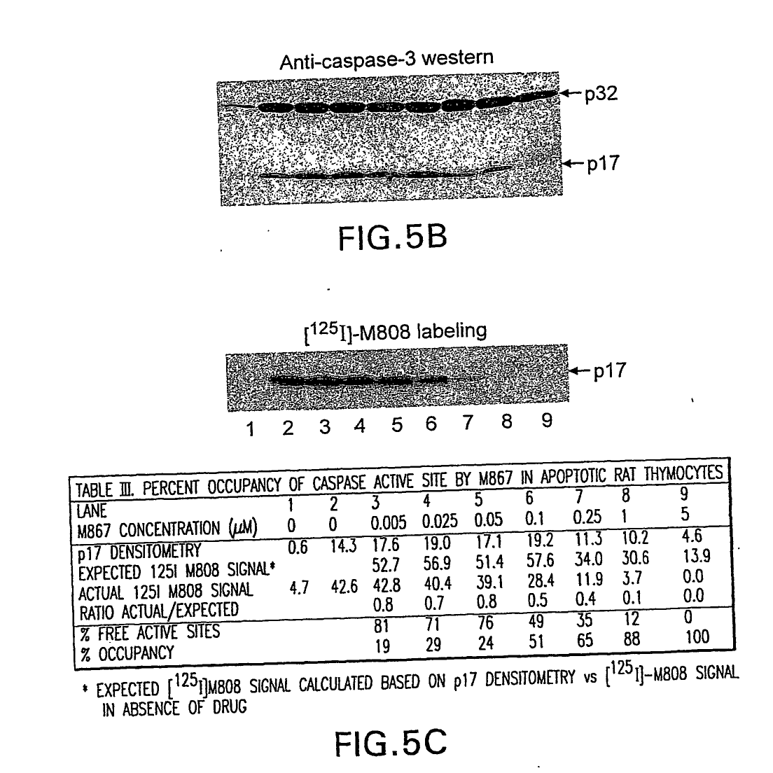

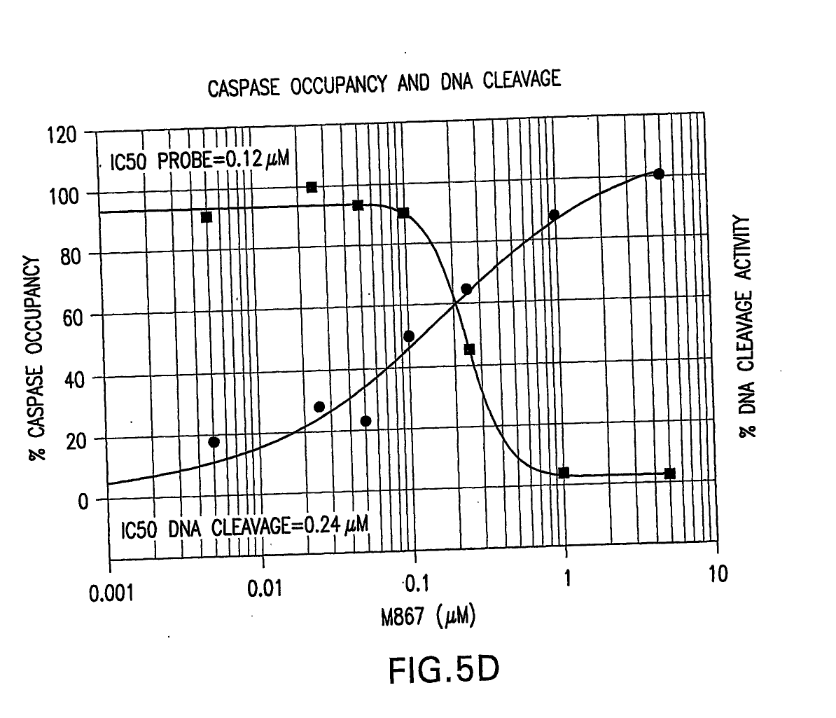

A similar experiment was performed in primary rat thymocytes that spontaneously die by apoptosis when put in culture. The study of several caspase inhibitors uncovered a discrepancy between the IC₅₀s determined by DNA fragmentation and by spectrin cleavage, in cultured thymocytes and in an in vivo model of cellular injury (N. Methot et al., in press). Three-fold more M867 is required to inhibit DNA fragmentation compared to spectrin cleavage (IC₅₀ of 0.27 and 0.09 μM, respectively, p<0.01). We speculated that the inhibition of DNA fragmentation required a greater fractional inhibition of caspase-3 by M867. To test this hypothesis directly, the percentage of caspase-3 occupied by M867 in cultured thymocytes was measured by [¹²⁵I]-M808 labeling. Cleaved caspase-3 and DNA fragmentation were assessed in parallel. As observed previously, M867 inhibited DNA fragmentation in cultured rat thymocytes with an IC₅₀ of 0.24 μM (FIG. 5), and had minimal impact on caspase-3 cleavage except at the highest concentration (FIG. 5B). Incubation of thymocytes with M808 showed occupancy of caspase by M867, with an IC₅₀ of 0.14 μM. Five independent experiments confirm that the IC₅₀ determined with M808 is significantly lower than the DNA fragmentation IC₅₀ (0.13+/−0.025 μM vs 0.27+/−0.045 μM, p=0.03), but is not statistically different from the IC₅₀ determined by spectrin cleavage. With the vast majority of labeled p17 caspase being accounted for by caspase-3 in these cells, we conclude that [¹²⁵I]-M808 labeling in whole cells reflects the true amount of active caspase-3. We also determined the actual percentage of caspase occupied by M867. (Table III). In FIG. 5A, an explanation of the calculations used to termined the percentage of capase active site occupancy is provided.

M808 Detects Active Caspases In Vivo

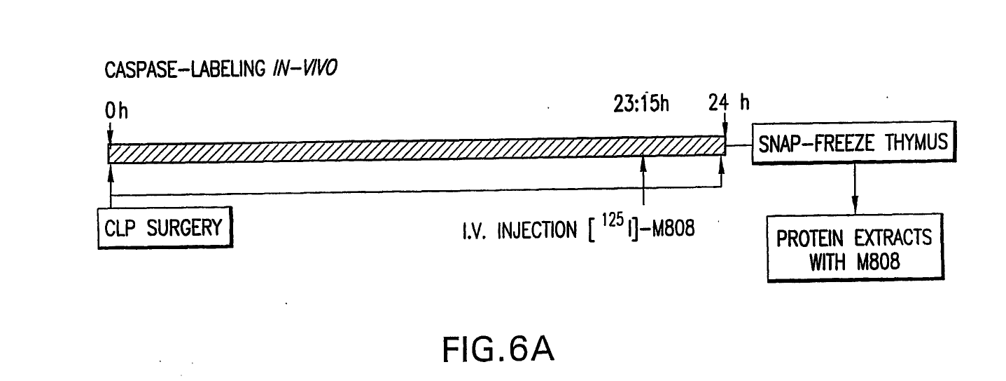

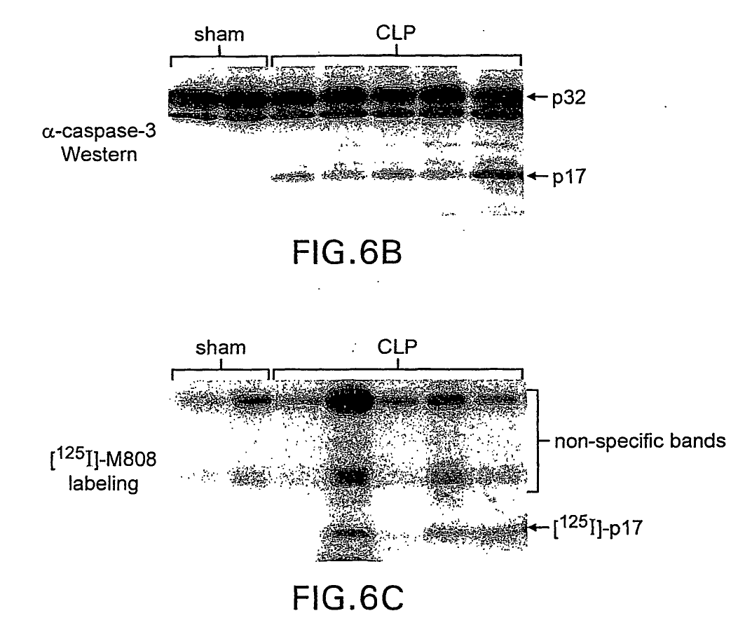

Next, we determined whether [¹²⁵I]-M808 could detect active caspases in vivo. Cecal ligation and puncture in mice results in peritonitis an activation of caspases is tissues such as the spleen, thymus, and gut. Caspase-3 is the principal caspase activated in the thymus, with a peak of activity between 16 and 24 h post-CLP (N.M., unpublished data). Peritonitis was induced for 23 hours by CLP in mice, and [¹²⁵I]-M808 was injected intravenously in animals 1 hour prior to euthanasia (FIG. 6A). Western blot on thymi protein extract reveals the presence of p17 caspase-3 in all CLP but not sham-operated animals (FIG. 6B). Similarly, a radiolabeled p17 protein was seen only in CLP but not in sham animals (FIG. 6C). Thus, [¹²⁵I]-M808 can detect active caspases in vivo. However, in contrast to protein extract and whole cell labeling, the intensity of the p17 signal was not proportional to the amount of p17 caspase-3 present. This was equally true for non-specifically labeled thymus proteins, despite equal gel loading. This discrepancy may be due to the variable accessibility of the probe to the injured thymus during sepsis, as this condition is known to cause organ hypoperfusion. As such, even though [¹²⁵I]-M808 detects active caspases in vivo, its usefulness is limited to systems where tissues are equally well perfused in all animals. However, an ex vivo on thymocytes from septic animals dosed with M867 will enable caspase active occupancy determination (see FIG. 5).

What is claimed is:

1. A compound represented by Formula I: 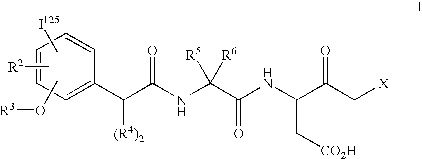
or a salt, ester or hydrate thereof, wherein:
X is halo, or X is O—W-Z, wherein W is a bond, —CH₂—, (O)— or (O)CH₂—;
Z is selected from the group consisting of:
(1) H,
(2) C₁₋₁₁alkyl,
(3) C₃₋₁₁cycloalkyl or a benzofused analog thereof,
(4) phenyl or naphthyl, and
(5) HET¹, wherein HET¹ represents a 5- to 10-membered mono- or bicyclic, aromatic or non-aromatic ring, or a benzofused analog thereof, containing 1-3 heteroatoms selected from O, S and N,
groups (2), (3) and (5) above are optionally substituted with 1-2 oxo groups,
groups (2)-(5) above are further optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₄alkylthio,
(g) C₃₋₆cycloalkyl,
(h) phenyl or naphthyl,
(i) phenoxy,
(j) benzyl,
k) benzyloxy, and
(l) a 5 or 6-membered aromatic or non-aromatic ring containing from 1-3 heteroatoms selected from O, S and N,
groups (d)-(g) above are optionally substituted with oxo and 1-3 substituents independently selected from halo and C₁₋₄alkoxy,
groups (h)-(l) above are optionally substituted with 1-3 substituents independently selected from halo and C₁₋₄alkyl, and
group (4) is further optionally substituted up to its maximum with halo groups;
R² is selected from the group consisting of:
(1) H,
(2) halo,
(3) hydroxy,
(4) nitro,
(5) cyano,
(6) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo or carboxy groups and further optionally substituted with 1-3 substituents independently selected from the group consisting of:
(a) halo,
(b) hydroxy
(c) cyano,
(d) C₁₋₄alkoxy,
(e) —NHR⁷, wherein R⁷ is independently H or C₁₋₅alkyl,
(f) —S(O)₀₋₂C₁₋₄alkyl, and
(g) HET², wherein HET² represents a 5- to 7-membered aromatic or non-aromatic ring containing 14 heteroatoms selected from O, S and NR⁸, wherein R⁸ is independently H or C₁₋₅alkyl, said HET² being optionally substituted with oxo and further optionally substituted with 1-2 substituents independently selected from halo and C₁₋₄alkyl, said C₁₋₄alkyl being optionally substituted with 1-3 halo groups,
(7) phenoxy or —S(O)₀₋₂phenyl,
(8) benzyloxy or —S(O)₀₋₂benzyl,
(9) benzoyl,
(10) phenyl or naphthyl,
(11) —O-HET² or —HET², said HET² being optionally substituted with oxo and further optionally substituted as defined below, and
(12) HET³, wherein HET³ is a 5- or 6-membered aromatic or non-aromatic ring, or a benzofused analog thereof, containing from 1 to 4 heteroatoms selected from O, S and N, said HET³ being optionally substituted with oxo and further optionally substituted as defined below,
groups (7)-(12) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups;
R³ is phenyl or C₁₋₁₀alkyl, said C₁₋₁₀alkyl optionally substituted with 1-2 oxo or carboxy groups and further optionally substituted with 1-3 substituents independently selected from the group consisting of:
(a) halo,
(b) hydroxy
(c) cyano,
(d) C₁₋₄alkoxy,
(e) —NHR⁷, wherein R⁷ is independently H or C₁₋₅alkyl,
(f) —S(O)₀₋₂C₁₋₄alkyl, and
(g) HET², wherein HET² represents a 5- to 7-membered aromatic or non-aromatic ring containing 1-4 heteroatoms selected from O, S and NR⁸, wherein R⁸ is independently H or C₁₋₅alkyl, said HET² being optionally substituted with oxo and further optionally substituted with 1-2 substituents independently selected from halo or C₁₋₄alkyl, said C₁₋₄alkyl being optionally substituted with 1-3 halo groups,
each R⁴ is independently selected from the group consisting of: H, halo, hydroxy, C₁₋₆alkyl and C₁₋₄alkoxy, said C₁₋₆alkyl and C₁₋₄alkoxy being optionally substituted with oxo and further optionally substituted with 1-3 halo groups; and
R⁵ is selected from the group consisting of: H, phenyl, naphthyl, C₁₋₆alkyl optionally substituted with OR¹² and 1-3 halo groups, and C₅₋₇ cycloalkyl optionally containing one heteroatom selected from O, S and NR¹³,
wherein R¹² is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy, and R¹³ is H or C₁₋₄alkyl optionally substituted with 1-3 halo groups; and
R⁶ represents H;
or in the alternative, R⁵ and R⁶ are taken in combination and represent a ring of 4-7 members, said ring optionally containing one heteroatom selected from O, S and NR¹³.

2. The compound according to claim 1 wherein X is halo.

3. The compound according to claim 1 wherein X is —O—W-Z.

4. The compound according to claim 3 wherein Z is selected from the group consisting of:
(1) C₁₋₁₁alkyl,
(2) C₃₋₁₁cycloalkyl or a benzofused analog thereof, and
(3) phenyl or naphthyl,
wherein groups (1)-(3) above are optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₄alkylthio,
(g) C₃₋₆cycloalkyl,
(h) phenyl or naphthyl,
(i) phenoxy,
(j) benzyl and
(k) benzyloxy.

5. The compound according to claim 1 wherein R³ is methyl.

6. The compound according to claim 1 wherein R² and each R⁴ are hydrogen.

7. The compound according to claim 1 wherein R⁵ is selected from the group consisting of: C1-6alkyl, phenyl and naphthyl.

8. The compound according to claim 1 wherein:
X is halo or —O—W-Z;
W is a bond, —CH₂—, C(O)— or —C(O)CH₂—;
Z is selected from the group consisting of:
(1) C₁₋₆alkyl, optionally substituted with 1-3 halo groups,
(2) C₃₋₁₁cycloalkyl or a benzofused analog thereof, and
(3) phenyl or naphthyl, optionally substituted with 1-3 groups independently selected from halo or C₁₋₄alkyl,
R³ is methyl, ethyl or phenyl;
R² and each R⁴ are hydrogen;
R⁵ is selected from the group consisting of: C₁₋₆alkyl, C₅₋₇cycloialkyl, phenyl and naphthyl; and
R6 is hydrogen.

9. A compound of Formula II 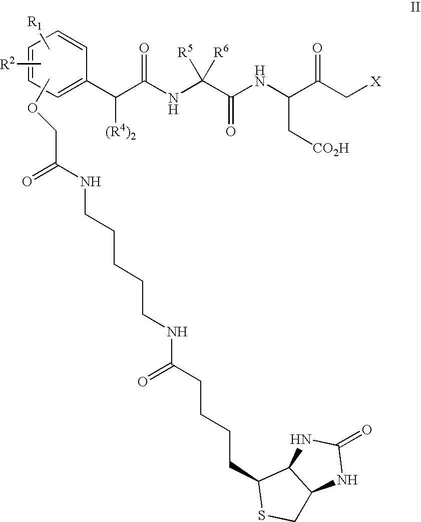
or a salt, ester or hydrate thereof, wherein:
X is halo;
R¹ and R² are each independently selected from the group consisting of:
(1) H,
(2) halo,
(3) hydroxy,
(4) nitro,
(5) cyano,
(6) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo or carboxy groups and further optionally substituted with 1-3 substituents independently selected from the group consisting of:
(a) halo,
(b) hydroxy
(c) cyano,
(d) C₁₋₄alkoxy,
(e) —NHR⁷, wherein R⁷ is independently H or C₁₋₅alkyl,
(f) —S(O)₀₋₂C₁₋₄alkyl, and
(g) HET², wherein HET² represents a 5- to 7-membered aromatic or non-aromatic ring containing 14 heteroatoms selected from O, S and NR⁸, wherein R⁸ is independently H or C₁₋₅alkyl, said HET² being optionally substituted with oxo and further optionally substituted with 1-2 substituents independently selected from halo and C₁₋₄alkyl, said C₁₋₄alkyl being optionally substituted with 1-3 halo groups,
(7) phenoxy or —S(O)₀₋₂phenyl,
(8) benzyloxy or —S(O)₀₋₂benzyl,
(9) benzoyl,
(10) phenyl or naphthyl,
(11) —O-HET² or —S-HET², said HET² being optionally substituted with oxo and further optionally substituted as defined below, and
(12) HET³, wherein HET³ is a 5- or 6-membered aromatic or non-aromatic ring, or a benzofused analog thereof, containing from 1 to 4 heteroatoms selected from O, S and N, said HET³ being optionally substituted with oxo and further optionally substituted as defined below,
groups (7)-(12) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups;
R³ is C₁₋₁₀alkyl, optionally substituted with 1-2 oxo or carboxy groups and further optionally substituted with 1-3 substituents independently selected from the group consisting of:
(a) halo,
(b) hydroxy
(c) cyano,
(d) C₁₋₄alkoxy,
(e) —NHR⁷, wherein R⁷ is independently H or C₁₋₅alkyl,
(f) —S(O)₀₋₂C₁₋₄alkyl, and
(g) HET², wherein HET² represents a 5- to 7-membered aromatic or non-aromatic ring containing 14 heteroatoms selected from O, S and NR⁸, wherein R⁸ is independently H or C₁₋₅alkyl, said HET² being optionally substituted with oxo and further optionally substituted with 1-2 substituents independently selected from halo or C₁₋₄alkyl, said C₁₋₄alkyl being optionally substituted with 1-3 halo groups,
each R⁴ is independently selected from the group consisting of: H, halo, hydroxy, C₁₋₆alkyl and C₁₋₄alkoxy, said C₁₋₆alkyl and C₁₋₄alkoxy being optionally substituted with oxo and further optionally substituted with 1-3 halo groups; and
R⁵ is selected from the group consisting of: H, phenyl, naphthyl, C₁₋₆alkyl optionally substituted with OR¹² and 1-3 halo groups, and C₅-7 cycloalkyl optionally containing one heteroatom selected from O, S and NR¹³,
wherein R¹² is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy, and
R¹³ is H or C₁₋₄alkyl optionally substituted with 1-3 halo groups; and
R⁶ represents H;
or in the alternative, R⁵ and R⁶ are taken in combination and represent a ring of 47 members, said ring optionally containing one heteroatom selected from O, S and NR¹³.

10. The compound according to claim 9 wherein:
R¹ is selected from the group consisting of:
(1) halo,
(2) C₁₋₄alkyl or C₁₋₄alkoxy, each optionally substituted with oxo and 1-3 halo groups, and
(3) HET³, wherein HET³ is a 5- or 6-membered aromatic or non-aromatic ring, or a benzofused analog thereof, containing from 1 to 4 heteroatoms selected from O, S and N, and optionally substituted with 1-2 substituents independently selected from halo and C₁₋₄alkyl, said C₁₋₄alkyl being optionally substituted with 1-3 halo groups;
R² and each R⁴ are hydrogen;
R⁵ is selected from the group consisting of: C₁₋₆alkyl, phenyl and naphthyl; and
R6 is hydrogen.

11. The compound according to claim 10 wherein HET³ is 1,2,4-oxadiazole, optionally substituted with C₁₋₄alkyl.

12. A method for detecting active caspase-3 in cells or tissues of a mammal comprising contacting said cells or tissues with a compound of claim 1 and detecting active caspase-3.

13. A method for detecting active caspase-3 in cells or tissues of a mammal comprising contacting said cells or tissues with a compound of claim 9 and detecting active caspase-3.

14. A method for determining the caspase-3 active site occupancy of a sample reversible caspase-3 inhibitor in an animal model of cellular injury comprising:
1) administering to said animal said sample reversible caspase-3 inhibitor;
2) euthanizing said animal and extracting said injured cells;
3) contacting said injured cells ex vivo with a compound according to claim 1;
4) detecting the amount of said compound to determine the number of caspase-3 free active sites; and
5) comparing said number of caspase-3 free active sites to the total measure of active caspases to determine the caspase-3 active site occupancy.

15. A method for determining the caspase-3 active site occupancy of a sample reversible caspase-3 inhibitor in a cell culture comprising:
1) contacting said cell culture with a sample reversible caspase-3 inhibitor;
2) contacting said cell culture with a compound according to claim 1;
3) detecting the amount of said compound to determine the number of caspase-3 free active sites; and
4) comparing said number of caspase-3 free active sites to the total measure of active caspases to determine the caspase-3 active site occupancy.

16. A kit for detecting active caspase-3 in cells or tissues of a mammal comprising a compound of claim 1.

17. A kit for detecting active caspase-3 in cells or tissues of a mammal comprising a compound of claim 9.

18. The compound according to claim 1 which is 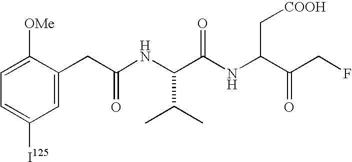
or a salt, ester or hydrate thereof.

19. A compound according to claim 1 which is selected from the following table: 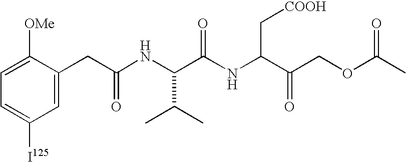 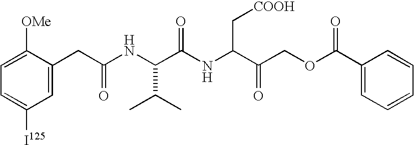 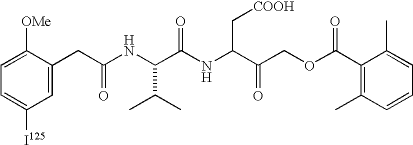 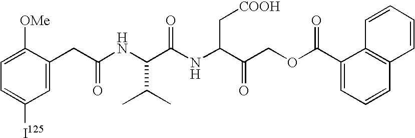 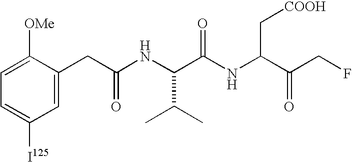 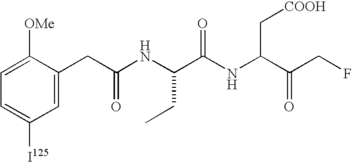 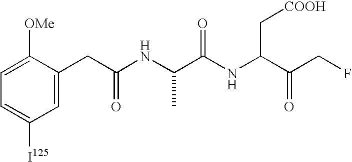 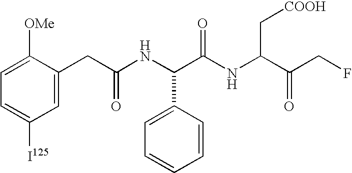 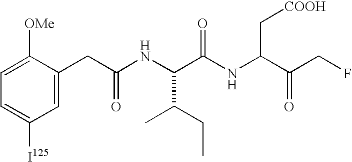 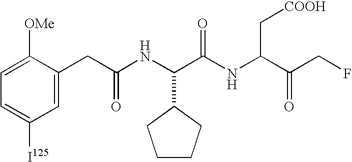 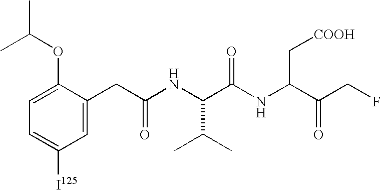 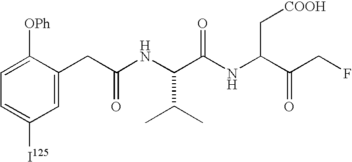 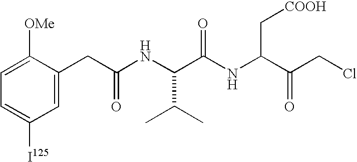 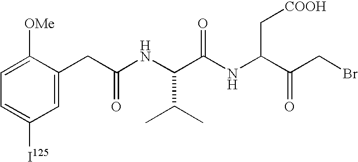 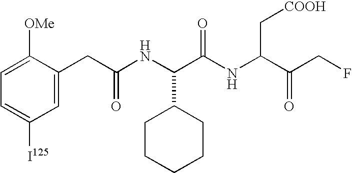
or a salt, ester or hydrate of any of the above.

20. A compound of any one of claims 1 to 11, 18 or 19 for use in detecting active caspase-3 in cells or tissues of a mammal.

21. A compound of any one of clams 1 to 11, 18 or 19 for use in determining the caspase-3 active site occupancy of a sample reversible capase-3 inhibitor in an animal model of cellular injury.
